SEQUENCE LISTING (20 sequences)

SEQ ID NO: 1 - DNA, 20 bp, Artificial Sequence
  Synthetic Artificial Construct
gcggggcggtgcgtgactac

SEQ ID NO: 2 - DNA, 24 bp, Artificial Sequence
  Synthetic Artificial Construct
aggcaagggggaagagaaggatgt

SEQ ID NO: 3 - DNA, 19 bp, Artificial Sequence
  Synthetic Artificial Construct
atcactggagcagggaagt

SEQ ID NO: 4 - DNA, 19 bp, Artificial Sequence
  Synthetic Artificial Construct
gctactacgtttcttatct

SEQ ID NO: 5 - DNA, 20 bp, Artificial Sequence
  Synthetic Artificial Construct
ccgcgtggagtgacctctaa

SEQ ID NO: 6 - DNA, 23 bp, Artificial Sequence
  Synthetic Artificial Construct
ggtggaattggaactgagcactt

SEQ ID NO: 7 - DNA, 20 bp, Artificial Sequence
  Synthetic Artificial Construct
acagccagtacttcaagacc

SEQ ID NO: 8 - DNA, 20 bp, Artificial Sequence
  Synthetic Artificial Construct
ctgtgtcagcacgcacgtta

SEQ ID NO: 9 - DNA, 21 bp, Artificial Sequence
  Synthetic Artificial Construct
tcttctacacacccatgtccc

SEQ ID NO: 10 - DNA, 18 bp, Artificial Sequence
  Synthetic Artificial Construct
cacctagtcacgacgtgg

SEQ ID NO: 11 - DNA, 18 bp, Artificial Sequence
  Synthetic Artificial Construct
acctagactcccgccgtg

SEQ ID NO: 12 - DNA, 18 bp, Artificial Sequence
  Synthetic Artificial Construct
cttgaacccgcgtctgta

SEQ ID NO: 13 - DNA, 20 bp, Artificial Sequence
  Synthetic Artificial Construct
cgcatactactgcctctccc

SEQ ID NO: 14 - DNA, 20 bp, Artificial Sequence
  Synthetic Artificial Construct
cagcagcgcagggcatcaaa

SEQ ID NO: 15 - DNA, 20 bp, Artificial Sequence
  Synthetic Artificial Construct
ctgcatcgtcctggctttgg

SEQ ID NO: 16 - DNA, 20 bp, Artificial Sequence
  Synthetic Artificial Construct
tgcagccagctttgcgttcc

SEQ ID NO: 17 - DNA, 20 bp, Artificial Sequence
  Synthetic Artificial Construct
ggatctgctcacatagtcac

SEQ ID NO: 18 - DNA, 20 bp, Artificial Sequence
  Synthetic Artificial Construct
ccaagtaggatgtgccagta

SEQ ID NO: 19 - DNA, 20 bp, Artificial Sequence
  Synthetic Artificial Construct
acctgacagactacctcatg

SEQ ID NO: 20 - DNA, 20 bp, Artificial Sequence
  Synthetic Artificial Construct
atcgtactcctgcttgctga

(19) United States
(12) Patent Application Publication
(10) Pub. No.: US 2013/0034525 A1
(43) Pub. Date: Feb. 7, 2013

(54) BETA ISELETS-LIKE CELLS DERIVED FROM WHOLE BONE MARROW

(71) Applicants: John S. Yu, Los Angeles, CA (US); Anna Milanesi, Beverly Hills, CA (US)

(72) Inventors: John S. Yu, Los Angeles, CA (US); Anna Milanesi, Beverly Hills, CA (US)

(73) Assignee: CEDARS-SINAI MEDICAL CENTER, Los Angeles, CA (US)

(21) Appl. No.: 13/566,519

(22) Filed: Aug. 3, 2012

Related U.S. Application Data
(60) Provisional application No. 61/514,612, filed on Aug. 3, 2011.

(51) Int. Cl. A61K 35/28; A61P 3/10; C12N 15/861; A61K 35/12; C12N 5/0775; C12N 15/86

(52) U.S. Cl. 424/93.7; 435/377; 435/456

(57) ABSTRACT

The invention provides for methods of producing pancreatic precursor cells and insulin-producing cells from bone marrow. In various embodiments, bone marrow derived stem cells are differentiated into pancreatic precursor cells and insulin-producing cells. In various embodiments, bone marrow derived stem cells are artificially induced to express VEGF and/or PDX-1. These cells can be used to treat or ameliorate diabetes or symptoms of diabetes.

### CROSS-REFERENCE TO RELATED APPLICATIONS

This application claims priority under 35 U.S.C. §119(e) to U.S. Provisional Application No. 61/514,612 filed on Aug. 3, 2011, the entirety of which is incorporated by reference.

### FIELD OF INVENTION

This invention relates to cell based therapeutic treatments for diabetes.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Understanding the mechanisms of β-cell homeostasis and regenerative repair is crucial not only to provide new insights on diabetes mellitus pathogenesis, but also to address potential therapeutic application. Islet cell transplantation may be a promising approach for cell replacement in type 1 diabetes. However, islet availability for allogeneic transplantation is limited (Lakey et al. 2006, Shapiro et al. 2006). In addition, understanding the origin of new β-cells in adults may help devise ways of slowing down progression of type 2 diabetes.

The mechanisms by which β-cell mass is maintained in adulthood are not yet known and the origin of new β-cells in adults is the focus of intensive research. The literature is replete with studies trying to characterize a putative pancreatic progenitor cell in and outside the pancreas that could explain β-cell neogenesis in adult life. Should such a cell be identified, it could be used as a potential source of cell therapy. It has been demonstrated that replacement of β-cells in the post-natal period precedes both the proliferation of existing mature β-cells (Dor et al. 2004), and the neogenesis of new β-cells from progenitor epithelial cells. It has been proposed that these adult pancreatic progenitor cells reside in the epithelium of the pancreatic duct (Bouwens & Kloppel 1996, Bonner-Weir 2000, 2001), inside islets (Zulewski et al. 2001) and in the bone marrow (Janus et al. 2003).

Nestin is an intermediate filament protein known as a marker of neuroepithelial stem cells, because it is expressed transiently in early developmental stages as well as in the process of tissue regeneration in various organs (Lendahl et al. 1990, Morshead et al. 1994, Matsuoka et al. 2002). Immunohistochemical studies showed the presence of nestin positive cells within the islet, the acinar and the ductal compartment of the prenatal and newborn pancreas (Hunziker & Stein 2000, Zulewski et al. 2001, Kim et al. 2004, Yashpal et al. 2004). The developmental progression and the change in nestin immunoreactivity throughout the fetal pancreas to postnatal transition suggest that nestinpositive cells are probably a population of progenitor cells within the pancreas (Yashpal et al. 2004). Moreover, Zulewski et al. (2001) described the existence of a distinct population of cells within islets and in a focal region of pancreatic ducts and exocrine pancreas expressing nestin that can proliferate and differentiate into pancreatic, exocrine, ductal and endocrine cells in culture. A recent publication showed that suppression of nestin expression in embryonic stem cells by gene silencing reduced endodermal and pancreatic transcription factor expression (Kim et al. 2010). During embryonic development, neural and islet cells express a subset of markers in common. Developing islet cells express several neuronalspecific markers, such as synaptophysins, nerve-specific enolasis (Alpert et al. 1988), and transcription factor genes such as Isl-1, Pax6, Pax4, b2/NeuroD, and IDX1 (Reynolds & Weiss 1996, Madsen et al. 1997, Sander & German 1997).

Therefore, these data suggest that nestin could be a common marker of a precursor stem cell for both neuronal and islet cell types. The inventors' group previously demonstrated that a subpopulation of nestin-expressing cells, isolated from bone marrow is able to generate cellular spheres similar to neurospheres derived from brain neural stem cells. These cells from bone marrow could differentiate into all three neural phenotypes (neurons, astrocytes, and oligodendrocytes) in vitro and in vivo (Kabos et al. 2002, Zeng et al. 2007). Recent studies showed that bone marrow-derived stem cells could reverse the hyperglycemic phenotype in a diabetic animal model but the mechanism behind the rescue or regeneration of pancreatic islets is still debated (Hasegawa et al. 2007, Xu et al. 2007, Gao et al. 2008, Zhao et al. 2008).

Diseases like diabetes are largely caused by a breakdown in cell function or by cell death. The major issue of diabetes is an inability to control the level of glucose (sugar) in the blood. Insulin therapy has saved the lives of many type 1 and type 2 diabetes mellitus patients. However, 50% of diabetics develop chronic diabetes-related complications that appear years after the onset of diabetes (including blindness, renal failure, myocardial infarction, and non-traumatic amputation).

Despite studies showing that strict blood glucose control decreases the incidence of secondary complications of diabetes, euglycemia is difficult to achieve with any current method of exogenous insulin replacement. Although transplantation of the whole pancreas or islets of Langerhans demonstrates the physiologic advantages of transplanting insulin-producing cells over insulin administration, these approaches are far from perfect. Ideally there are two possible solutions: identifying the perfect “surrogate β-cell” to be used for cell therapy or inducing regeneration of endogenous damaged β-cells.

Stem cells from bone marrow offer an attractive source of stem cells as alternative to pancreas and pancreatic islets transplantation for curative and definitive treatment of insulin dependent diabetes. They are already proved to be safe in clinical trial and they can be obtained with relative ease from each patient, allowing potential circumvention of allograft rejection. Previous work suggested that the mouse BMSCs spontaneously differentiate into endocrine pancreas cells in vivo. In recent reports, BMSCs injected into the circulation of diabetic animal has been shown to partially/totally reverse the diabetic phenotype and improve glucose control, but with very poor direct β-cell differentiation, leading to other possible roles of BMSCs in the pancreatic islet regeneration. According, additional treatment methods are needed in the art.

Encouragingly, as further described herein the introduction of transcription factor genes into cultured human BMSCs was able to activate a number of genes related with development and function of β-cells. Moreover, the forced expression of Pdx1 gene by a virus vector in human BMSCs showed the activation of gene expression of all four islets hormones and also enhancement of significant insulin content.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method, comprising: providing a nestin-positive bone marrow stem cell (BMSC); culturing the nestin-positive BMSC in a first culture medium for chromatin remodeling by contacting the cell with 5-azacytidine (5-AZA) and contacting the cell with trichostatin (TSA); culturing the cell in a second culture medium for cell induction; culturing the cell in a third culture medium to differentiate the cell; culturing the cell in a forth culture medium to mature the cell into a pancreatic precursor cell or an insulin-producing cell. In various embodiments, the insulin-producing cell is an insulin producing β-cell or a β-islet cell.

In various embodiments, the first culture medium can comprise one or more agents selected from the group consisting of KO-DMEM, β-mercaptoethanol, L-glutamine, non-essential amino acid, BSA, N2 supplement, B27 supplement, heparin, bFGF, EGF, and combinations thereof. In various embodiments, the second medium can comprise one or more agents from the first culture medium and an agent selected from the group consisting of DMEM, low glucose, ITS, RA and combinations thereof. In various embodiments, the third medium can comprise one or more agents from the first culture medium and an agent selected from the group consisting of DMEM, high glucose and combinations thereof. In various embodiments, the fourth medium can comprise one or more agents from the first culture medium and an agent selected from the group consisting of DMEM, high glucose, nicotinamide, and combinations thereof. In various embodiments, the fourth culture medium does not contain bFGF or EGF.

Various embodiments of the present invention provide for a method, comprising: providing a bone marrow stem cell (BMSC); and transfecting the BMSC with one or more virus vectors encoding PDX1, VEGF, or both to produce a VEGF and/or PDX1 expressing BMSC. In various embodiments, the virus can be an adenovirus.

In various embodiments, the BMSC can be obtained by: providing bone marrow aspirate; culturing a nucleated cell from the bone marrow aspirate; separating a non-adherent cell from an adherent cell; culturing the adherent cell; harvesting a bone marrow stem cell; and expanding the bone marrow stem cell. In various embodiments, the nucleated cell from the bone marrow aspirate may be cultured in a medium comprising a component selected from the group consisting of Afla-Men, fetal bovine serum, glutamine, penicillin, streptomycin and combinations thereof.

Various embodiments of the present invention provide for a method, comprising: providing a bone marrow stem cell (BMSC) expressing VEGF, a BMSC expressing PDX1 or a BMSC expressing both VEGF and PDX1; and administering the BMSC to a subject in need of treatment for diabetes or ameliorating a symptom of diabetes to treat diabetes or ameliorate the symptom of diabetes. In various embodiments, administering can be via intravenous injection of BMSC expressing of VEGF and/or PDX1 into the circulation of the subject.

In various embodiments, the BMSC expressing VEGF, the BMSC expressing PDX1 or the BMSC expressing both VEGF and PDX1 can be produced by: isolating a BMSC from a bone marrow sample from the subject; and artificially increasing the expression of VEGF and/or PDX1 in the BMSC.

Various embodiments of the present invention provide for a method, comprising: providing a pancreatic precursor cell or an insulin-producing cell differentiated from a bone marrow stem cell (BMSC); and administering the pancreatic precursor cell or the insulin-producing cell differentiated from a BMSC to a subject in need of treatment for diabetes or ameliorating a symptom of diabetes to treat diabetes or ameliorate the symptom of diabetes. In various embodiments, administering can be via intra-hepatic or subcutaneous transplantation.

In various embodiments, the method can further comprise obtaining bone marrow from the subject; and producing the pancreatic precursor cell or the insulin-producing cell from the bone marrow. In various embodiments, the insulin-producing cell can be an insulin-producing β-cell or a β-islet cell.

In various embodiments, producing the pancreatic precursor cell or an insulin-producing cell from the bone marrow can be by: producing a nestin-positive bone marrow stem cell (BMSC) from the bone marrow; culturing the nestin-positive BMSC in a first culture medium for chromatin remodeling by contacting the cell with 5-azacytidine (5-AZA) and contacting the cell with trichostatin (TSA); culturing the cell in a second culture medium for cell induction; culturing the cell in a third culture medium to differentiate the cell; culturing the cell in a forth culture medium to mature the cells into a pancreatic precursor cell or an insulin-producing beta-cell.

In various embodiments, the first culture medium can comprise one or more agents selected from the group consisting of KO-DMEM, β-mercaptoethanol, L-glutamine, non-essential amino acid, BSA, N2 supplement, B27 supplement, heparin, bFGF, EGF, and combinations thereof. In various embodiments, the second medium can comprise one or more agents from the first culture medium and an agent selected from the group consisting of DMEM, low glucose, ITS, RA and combinations thereof. In various embodiments, the third medium can comprise one or more agents from the first culture medium and an agent selected from the group consisting of DMEM, high glucose and combinations thereof. In various embodiments, the fourth medium can comprise one or more agents from the first culture medium and an agent selected from the group consisting of DMEM, high glucose, nicotinamide, and combinations thereof.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts human BMSC-VEGF transplantation into chemically induced diabetic mice in accordance with various embodiments of the present invention. Panel 1A shows blood glucose level of control healthy mice (gray rectangle) versus STZ-induced diabetic mice (black diamond). Panel 1B shows blood glucose level of mice rescued from diabetes after hBMSC-VEGF treatment (Rescued, white diamond) versus unrescued mice (Unrescued, black rectangle). Weight in gram (g) (C) of control mice, diabetic mice (STZ) and diabetic mice treated with hBMSC-VEGF (hBMSC-VEGF) at the moment of stem cell transplantation (Day 0; blue bar) and after 6 weeks (day 42; purple bar) shows significant weight gain in the control group and in the hBMSC-VEGF treated group, but not significant weight gain in the diabetic group. In addition weight of control mice and hBMSC-VEGF treated mice is significantly higher than diabetic mice at 6 weeks. Survival analysis (D) shows 100% survival of hBMSC-VEGF treated mice (red line) at 6 weeks, compared with the significant reduced survival of diabetic (STZ) mice (blue line), p<0.05. Pancreas immunohistochemistry: H&E staining (E,F,G,H) and fluorescence staining for insulin (green) and glucagon (red) (I,J,K,L) of the pancreas of the control healthy mice (E,I), STZ-induced diabetic mice (F,J), hBMSC-VEGF rescued mice (G,K), and hBMSC-VEGF unrescued mice (H,L). Scale bar: 50 μm. *p<0.01, **p<0.001

FIG. 2 depicts engraftment of hBMSC-VEGF in the injured pancreas in accordance with various embodiments of the present invention. Fluorescence immunostaining for human β-2-microglobulin (green) shows engraftment of the hBMSC-VEGF in the pancreas of rescued mice (A). At higher magnification it is evident the differentiation to vascular and ductal structures in the pancreas (B). Fluorescence immunestaining for β-2-microglobulin shows more modest human stem cell engraftment in the pancreas of the unrescued mice (C). Panel 2D shows few cells in a pancreatic islet co-staining with human β-2-microglobulin (green) and insulin (red), and small vessel like structure inside the pancreas co-staining with β-2-microglobulin (green) and a smooth muscle actin (red). Scale bar: 50 μm. Panel E shows the percent of the pancreatic islets expressing human β2-microglobulin, and the percent of β-cells expressing human β2-microglobulin in the positive pancreatic islets (Right panel). Additionally, fluorescence immunostaining for human β-2-microglobulin (green) showed engraftment of the hBMSC-VEGF in the mesenchymal tissue outside the pancreas (F). At higher magnification it is evident the differentiation in vascular and ductal structure in vascular and adipose tissue in the mesenchymal outside the pancreas (G).

FIG. 3 depicts VEGF expression in the pancreatic islets in accordance with various embodiments of the present invention. Fluorescent staining for VEGF (red) and insulin (green) in the pancreatic islets of control mice (A-C), diabetic mice (STZ;D-F), and diabetic mice rescued by hBMSC-VEGF (G-I). Arrows (F) show only few cells co-expressing insulin and VEGF. Percentage of β-cells expressing VEGF in the pancreas of control mice, diabetic mice (STZ) and mice rescued by hBMSC-VEGF (J). Scale bar: 50 μm. **p<0.001.

FIG. 4 depicts human BMSC-PDX1 transplantation in chemically induced diabetic mice in accordance with various embodiments of the present invention. Blood glucose level (A) of temporary reversed mice (purple diamond, Rev), versus unrescued mice (black rectangle, Unrescued), versus control mice (white triangle), versus STZ-induced diabetic mice (green cross, STZ). Survival analysis (B) of diabetic mice injected with hBMSC-PDX1 (purple line) versus non injected diabetic mice (blue line) shows 100% survival at 6 weeks of mice treated with hBMSC-PDX1 compared with the significant lifespan reduction of diabetic non treated mice (p<0.05). Weight in gram (C) of control mice, diabetic mice (STZ), and diabetic mice treated with hBMSC-PDX1 at the moment of stem cell transplantation (day 0) and after 6 weeks (day 42) shows significant weight gain of control mice and hBMSC-PDX1 treated mice compared with diabetic mice. Immunoperoxidase staining for insulin in pancreases of control healthy mice (D), STZ-induced diabetic mice (E), temporary reversed (F), and unrescued mice (G). Immunofluorescent staining for human β2-microglobulin (green) shows engraftment of hBMSCs-PDX1 in the injured pancreas. A few hBMSCs co-express insulin (red) suggesting β-cell differentiation (H). Nuclei are stained with DAPI (Blue). Scale bar: 50 μm. *p<0.05, **p<0.001.

FIG. 5 depicts human BMSC transplantation in chemical induced diabetic mice in accordance with various embodiments of the present invention. Blood glucose level (A) of healthy control mice (gray rectangle), STZ-induced diabetic mice (black diamond, STZ) and STZ-induced diabetic mice after treatment with hBMSCs (white triangle, hBMSC). Blood glucose level (B) of healthy control mice (n=4; purple cross), STZ-induced diabetic (n=4, blue cross), STZ-induced diabetic mice after treatment with fibroblast expressing VEGF (n=4; purple square) or fibroblast expressing PDX1 (n=4; yellow triangle). Survival analysis (C) of diabetic mice injected with hBMSC (red line) versus non injected diabetic mice (blue line) versus control healthy mice (green line) shows lifespan reduction of mice injected with hBMSC (p<0.05), similar to non-injected diabetic mice. Weight in gram of diabetic mice treated with hBMSC is similar to untreated diabetic mice and significantly lower than healthy control mice (D). Moreover, no weight gain has been observed from the moment of stem cell transplantation (Day 0), to the end of the study (Day 42). Immunohistochemistry of pancreases from the diabetic mice treated with hBMSC; H&E staining shows reduced pancreatic islet size and altered morphology (E); fluorescence staining for insulin (green) and glucagon (red) shows decrease expression of insulin (F); fluorescence immunostaining for human β2-microglobulin (green) shows very poor engraftment of the hBMSCs in the diabetic pancreas (G). Scale bar: 50 μm. *p<0.05, **p<0.001

FIG. 6 depicts serum insulin and β-cell number in accordance with various embodiments of the present invention. Both healthy control mice and diabetic mice rescued by hBMSC-VEGF (hBM-VEGF-R) have significantly higher mouse insulin level (A) compared with diabetic mice (STZ), diabetic mice treated with hBMSC (hBM), diabetic mice unrescued by hBMSC-VEGF (hBM-VEGF-Ur), and diabetic mice treated with hBMSC-PDX1 (hBM-PDX1). There is no significant difference between mouse insulin level of healthy control mice and diabetic mice rescued by hBMSC-VEGF (hBM-VEGF-R). Human insulin is detected in 3 rescued mice treated with hBMSC-VEGF, 2 unrescued mice treated with hBMSC-VEGF, and 5 mice treated with hBMSC-PDX1 (B). Diabetic mice rescued by hBMSC-VEGF (hBM-VEGF-R) have significantly higher total insulin level (human and mouse insulin) (C) compared with diabetic mice (STZ), diabetic mice treated with hBMSC (hBM), diabetic mice unrescued by hBMSC-VEGF (hBM-VEGF-Ur), and diabetic mice treated with hBMSC-PDX1 (hBM-PDX1), and not significantly different from control healthy mice. Mice unrescued by hBMSC-VEGF and mice treated with hBMSC-PDX1 have significant higher total insulin level compared with diabetic mice and diabetic mice treated with hBMSC, but lower than control mice and mice rescued by hBMSC-VEGF. β-cell number is higher in the healthy control mice compared with other groups (D). Mice rescued by hBMSC-VEGF have significant higher β-cell number than diabetic mice, diabetic mice treated with hBMSC, diabetic mice unrescued by hBMSC-VEGF, and diabetic mice treated with hBMSC-PDX1, but significantly lower than healthy control mice. Diabetic mice unrescued by hBMSC-VEGF and treated with hBMSC-PDX1 have significantly higher β-cell number than diabetic mice and diabetic mice treated with hBMSC, but significantly lower than mice rescued by hBMSC-VEGF. *p<0.05, **p<0.01, ***p<0.001.

FIG. 7 depicts insulin/IGF receptor signaling pathway in the pancreas of diabetic mice, healthy control mice and mice rescued by hBMSC-VEGF: PCR array profile in accordance with various embodiments of the present invention. Panel 7A shows diabetic mice (n=4) compared with healthy control (n=4); panel 7B shows mice rescued by hBMSC-VEGF (n=4) compared with healthy control mice (n=4); panel 7C shows mice rescued by hBMSC-VEGF compared with diabetic mice. *p<0.05, **p<0.01, ***p<0.001.

FIG. 8 depicts insulin/IGF1 receptor/PI-3K/ATK pathway in the pancreas of diabetic mice, healthy control mice, and mice rescued byhBMSC-VEGF in accordance with various embodiments of the present invention. Immunostaining of pancreatic sections from control healthy mice (control), diabetic mice (STZ), and diabetic mice rescued by hBMSC-VEGF (hBMSC-VEGF) for AKT (green), insulin (red) and merge in yellow (A); PDX1 (green) and insulin (red) (B); p27-kip1 (green) and insulin (red) (C); Caspase 3 cleaved (red), insulin (green), and merge in yellow (D). Nuclei are stained with DAPI (blue). Percentage of β-cells expressing Caspase 3 cleaved (c-CASP3) in the pancreas of control mice (control), diabetic mice (STZ), and diabetic mice rescued by hBMSC-VEGF (E). Schematic summary of the Insulin receptor/PI-3K/AKT signaling pathway activated by treatment with hBMSC-VEGF: reduced apoptosis, increased β-cell differentiation and proliferation through PDX1 expression and down regulation of the cell cycle inhibitor p27-kip, and improved intra-islet angiogenesis through regulation of VEGF expression (F). **p<0.01.

FIG. 9 depicts differentiation steps of multipotent nestin-positive stem cell isolated from rat bone marrow (n-BMSC) in accordance with various embodiments of the present invention. Multipotent rat bone marrow stem cells formed neurospheres in vitro (A) (magnification ×10) and stained positive for nestin (B) (magnification ×20). (C) Schematic representation of multistep differentiation protocol of n-BMSC to pancreatic lineage (endocrine and ductal phenotype): chromatin remodeling with 5-AZA (5-azacytidine) and TSA (trichostatin) (step 1), induction with ITS and RA (step 2), differentiation (step 3), and maturation with nicotinamide (step 4).

FIG. 10 depicts expression of nestin and pancreatic transcription factors during in vitro n-BMSC differentiation in accordance with various embodiments of the present invention. (A-D) Immunofluorescence staining for nestin was positive (A) in the beginning and negative in the following differentiation step. (E-H) Immunofluorescence for PDX1 was negative in the beginning (A) and positive in the three following steps. (I-L) Immunofluorescence staining for PAX6 was negative in the beginning (A); positive in only few cells by step 2 (J), and in many cells by step 3 (K); negative again in the last step (L). Nuclei were counterstained with DAPI. Confocal microscopy, original magnification ×63 (A, F, G, and H); Fluorescent microscopy ×40 (K) and ×20 (J and L).

FIG. 11 depicts quantitative reverse transcription-PCR analysis of n-BMSC for Pdx1 and Ngn3 in accordance with various embodiments of the present invention. (A): Pdx1 and Ngn3 genes were not expressed in the beginning of pancreatic differentiation in vitro but were induced during step 2 of the differentiation protocol. DCT presents the difference of the cycle threshold between the assayed gene and the normalizing gene. (B) Variation of gene expression during in vitro differentiation. Values are fold change GS.D. (nZ3). *P<0.05; **P<0.01.

FIG. 12 depicts differentiation of n-BMSC in a mature pancreatic phenotype in accordance with various embodiments of the present invention. Immunofluorescence staining for CK19 (A and B) and for insulin (C and D) was negative until the third differentiation step and became positive in the last step (B and D). Dithizone staining for proinsulin (E and F) was also positive in the last differentiation step. Nuclei were counterstained with DAPI. Images of insulin staining visualized under confocal microscope (D-G) with detail of single-cell sectioning (H) showing the insulin granular cytoplasmatic pattern. Confocal microscopy, original magnification ×63 (B, D, G, and H); phase contrast microscopy, original magnification ×20 (E and F).

FIG. 13 depicts quantitative reverse transcription-PCR analysis of n-BMSC in accordance with various embodiments of the present invention. (A) Gene not expressed in the beginning of pancreatic differentiation in vitro and induced during step 3 of the differentiation protocol. DCT represents the difference between the cycle threshold of the assayed gene and that of the normalizing gene. (B) Variation of gene expression during in vitro differentiation. (C) Insulin production in response to glucose concentration: n-BMSCs were incubated in a buffer containing the indicating concentration of glucose. Values are fold change±S.D. (n=3). **P<0.01; ***P<0.005; ****P<0.0001.

FIG. 14 depicts blood glucose level after hBMSC transplantation in accordance with various embodiments of the present invention. (A): healthy control mice (rectangle), STZ-induced diabetes mice (black square), and STZ-induced diabetes mice after treatment with hBMSC (white triangle). Pancreases immunohistochemistry (B): H&E staining showed normal pancreatic islets number, size and morphology in the healthy control mice, compared with reduced size and altered morphology of the diabetic mice with (STZ+hBMSC) or without hBMSC treatment (STZ). Fluorescence staining for insulin (green) and glucagon (red) of the control pancreas, compared with the diabetic mice with (STZ+hBMSC) or without hBMSC (STZ). Scale bare: 50 μm.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus adult and newborn subjects, as well as fetuses, whether male or female, are intended to be including within the scope of this term.

“Treatment” and “treating,” as used herein refer to both therapeutic treatment and prophylactic or preventative measures, wherein the object is to prevent, slow down and/or lessen the disease even if the treatment is ultimately unsuccessful.

Described herein is a novel culture system in order to obtain insulin producing cells derived from nestin-positive bone marrow cells (n-BMSC). This procedure successfully induced n-BMSC to express lineage-specific transcription factor, in particular Pdx1, which has been shown to drive pancreatic differentiation.

The cells were pretreated with 5-AZA to inhibit DNA methylation, which has been recognized in important differentiation processes, such as osteoblast (Vaes et al. 2009), cardiomycyte (Tomita et al. 1999) and more recently pancreatic endocrine differentiation (Lefebvre et al. 2009). The cells were subsequently treated with TSA based on the belief that chromatin remodeling will enhance the induction and further differentiation toward pancreatic lineage, although not wishing to be bound by any particular theory. Acetylation or deacetylation of histone terminal domains can regulate gene expressions. Histone acetyltransferases and HDACs can loosen or compact chromatin structures and regulate cell proliferation and differentiation in various tissues (Zhang et al. 2002, Chang et al. 2004). As the acetylation status of nucleosomal histone modulates chromatin structure and epigenetically regulates gene expression, an inhibitor of both classes I and II HDACs such as TSA may be used to loosen the chromatin structure and more easily permit differentiation through epigenetic manipulation. Moreover, a recent study from Haumaitre et al. (2008) showed that HDAC inhibitors can modify the timing and the determination of pancreatic cell fate with inhibition of exocrine and enhancement of ductal and endocrine differentiation. In the experiment described herein, after exposure to 5-AZA and TSA, the cells lost nestin expression and after being cultured in a medium containing ITS and RA, they also turned on expression of mRNA encoding transcription factors such as Pdx1, NgN3 and Pax6, known to play important roles in the developmental program leading to the formation of pancreatic islets (Jensen 2004). Moreover, the expression of these transcription factors followed the timing consistent with the normal mammalian pancreatic development. PDX1 was expressed in the second step after exposure to RA and ITS medium and subsequently expression of both PAX6 and NGN3 were activated. In the last step, a more mature phenotype of ductal and endocrine pancreatic cells appeared. These cells lost the expression of PAX6, and upregulated PDX1, insulin, glucagon, GLUT2 and CK19.

Another important step in this differentiation protocol is the use of RA to induce differentiation of n-BMSC towards a pancreatic phenotype. In the experiment described herein, it was extremely important to time the exposure to RA, right after the cell treatment with TSA. RA has been shown to control multiple steps in the motor neuron differentiation in the ventral spinal cord (Novitch et al. 2003). In an experiment, Ostrom et al. (2008) showed that RA is present in the developing mouse and human pancreas and is required for pancreas development. Moreover, all-trans RA has been widely used to promote generation of PDX1-positive pancreatic progenitor cells from mouse embryonic stem cells (Micallef et al. 2005, Shi et al. 2005) and to induce pancreatic differentiation in human embryonic stem cells (D'Amour et al. 2006, Johannesson et al. 2009). In the experiment described herein, consistent with the previous observations, it is shown showed that RA induces expression of PDX1.

In the last step both ductal and endocrine phenotypes were present and this is probably because the activation of Pdx1 in the second step of the protocol, subsequently activated both the endocrine and the non-endocrine pathways. Pdx1 is well known for controlling early whole pancreas development.

The inventors believe, although not wishing to be bound by any particular theory, that a selected stem cell population from rat whole bone marrow highly expressing nestin could be a potential multipotent precursor stem cell able to differentiate to pancreatic cell lineage. This belief was based on the evidence that nestin is a neuroepithelial marker transiently expressed in early stages in many tissues, including pancreas, and that nestin-positive cells from pancreas can be differentiated in culture into endocrine, exocrine, and ductal cells (Zulewski et al. 2001).

The inventors demonstrated that epigenetic manipulation of n-BMSC, following a specific multistep protocol, can induce expression of transcription factors involved in the early pancreatic and endocrine specification in a step-wise fashion whose timing was consistent with normal pancreas development. Differentiation into a more mature phenotype, including ductal and insulin-producing cells was efficiently induced. It remains to be shown how long these cells remain in this differentiated state both in vitro and in vivo. In conclusion, the inventors describe a novel cellular system that can address these unmet needs, both for cell therapy and for mechanistic studies and drug discovery in the field of pancreatic islet neogenesis.

Moreover, the inventors were able to induce reversion of diabetes for genetic modification of bone marrow stem cells using PDX1 and VEGF gene. This is the first time that stem cell from bone marrow expressing VEGF has been used as possible treatment of type 1 Diabetes Mellitus. After transplantation in a diabetic animal model, the inventors observed de novo differentiation of human bone marrow stem cells into β-cells and for the first time in the literature the inventors report detectable level of human insulin, confirming a successful chimerism and a functional differentiation of human bone marrow stem cells in beta-cells.

Stem cell therapy may be a desirable alternative to pancreas and pancreatic islet transplantation, and stem cells from bone marrow represent an attractive source. However, the mechanism related to β-cell recovery required elucidation. Here, for the first time the inventors tested the hypothesis of de novo β-cell differentiation from hBMSCs versus endogenous β-cell regeneration mediated by hBMSCs, using the transient expression of PDX1 and VEGF.

In contrast to previous reports [3,7,8,9,10,11] hBMSCs alone were not able to reverse hyperglycemia in the inventors' animal model. This can be attributed to differences in stem cell populations, mouse strains, mouse models, and experimental designs among research groups. Some studies used hematopoietic stem cells from bone marrow [3,32], or mouse mesenchymal bone marrow stem cells [8,9,10,11]. In addition C57BL/6 were used for STZ-induced diabetes model [8,10,11], which can explain different outcomes in response to STZ, degree of diabetes, blood glucose levels and responses to treatment. Only one previous report [7] is the closest to the inventors' study: same type of stem cells, same cell delivery method (intracardiac injection) and same mouse strain. However the dose of STZ used was adjusted to produce nonlethal hyperglycemia and an improvement in blood glucose control was possible only with multiple injections of human cells. The higher glucose level and mortality of the inventors' mice in addition to a single stem cell injection can explain the different results.

The inventors achieved sustained recovery from diabetes following injection of hBMSCs overexpressing VEGF. The inventors observed an efficient engraftment of hBMSC-VEGF in the pancreas of the diabetic mice, and its successful differentiation into blood vessels and to lesser degree into β-cells. For the first time, the inventors reported detectable levels of human insulin confirming a successful chimerism in the mouse. However, the sustained near-normoglycemia remission was not fully supported by the low level of human insulin, and the low number of β-cells from hBMSCs-VEGF. The significantly higher level of mouse insulin in rescued mice suggested that the endogenous β-cell regeneration was the predominant mechanism behind the sustained clinical recovery. Taken together the de novo intra pancreatic angiogenesis from hBMSC-VEGF along with the endogenous activation of the insulin/IGF receptor signaling pathway strongly support regeneration and functional recovery of endogenous β-cells.

To highlight this concept, the inventors performed a parallel experiment using hBMSCs expressing PDX1. The previous report showed the possible direct differentiation of human hBMSCs into β-cells in vitro after transfection with a virus vector encoding PDX1[17], supporting the possible role of PDX1 to direct the differentiation of hBMSCs into insulin-producing cells in the inventors' in vivo model. Human BMSC-PDX1 could differentiate to β-cells in the diabetic pancreas with approximately the same efficiency of hBMSC-VEGF confirmed by similar detectable levels of serum human insulin and by immunohistochemistry. However, transplantation of hBMSC-PDX1 into diabetic mice resulted in only transient recovery. The overall efficiency of hBMSC-PDX1 engraftment and their differentiation into blood vessels were significantly lower than that from hBMSC-VEGF, which is correlated with the disparate clinical outcomes. VEGF-A has been known to play a key role in maintaining normal intra-islet vascularization [33,34]. In addition, VEGF is known to enhance proliferation, survival and differentiation of bone marrow mesenchymal stem cells [35]. Over expression of VEGF in BMSC increased revascularization and myocardial recovery after injury [36], and neutralizing anti-VEGF antibodies inhibited the BMSC-initiated angiogenic response in vivo [37]. Moreover the β-cell-specific VEGF-A deficient mouse showed the altered insulin secretion despite maintaining a normal β-cell mass [38] and bone marrow transplantation did not induce expansion of β-cell mass after STZ-induced diabetes compared with wild-type mice [39]. It has been reported that BMSCs improved revascularization and function of pancreatic islets after transplantation [40]. Bone marrow mesenchymal stem cells can not only promote endogenous angiogenesis [41], but directly differentiate into smooth muscle [42] and endothelial cell phenotypes [43] in vitro, and into functional vascular structures [44,45] and contribute in vivo to myocardial recovery after injury [46].

The pancreas of the mice rescued by hBMSC-VEGF showed upregulation of insulin receptor associated gene, such as Ins1, Igf2, Igfbp1 as well as Dok1, 2 and 3. Recent extensive studies have shown the importance of insulin regulating β-cell function [47]. The inventors' results showed that Insulin/IGF receptor coupling with insulin receptor soluble (IRS) proteins activated the downstream effector pathway PI-3K. Several gene targets in the PI-3K pathway were upregulated including Adra1d, G6 pc, G6 pc2, and Serpine 1 in the rescued group. In contrast, Grb2, generally thought to affect Ras and mitogen-activated protein kinase signaling, was significantly downregulated in the rescued mice. Consistent with a previous report [48], the expression of Jun was increased in the diabetic pancreas while it was significantly downregulated in the rescued mice. Insulin/IGF receptor/PI-3K signaling mediates several pathways related to proliferation and anti-apoptosis in most mammalian cells including pancreatic islets [49]. AKT is a critical mediator of the Insulin/IGF receptor/PI-3K pathway and overexpression of active AKT1 in the mouse β-cells significantly increased β-cell size and total islet mass [50]. The inventors showed a significant decrease of AKT expression in the pancreatic islets of diabetic mice, compared with control and rescued mice. Interestingly, pattern of AKT distribution was mostly on the cell membrane of the β-cells of the healthy control mice, while it was highly expressed in both cell membrane and cytoplasm of the β-cells in the rescued mice. It is well known that AKT activation takes place on the cell membrane [51]. On the other hand, it has been reported that translocation of AKT in the cytoplasm and nucleus after stimulation with growth factors such us insulin and IGF1 could mediate potential anti-apoptotic mechanisms [52,53]. Activation of Insulin/IGF signaling through PI-3K/ATK pathway could induce reduction of apoptosis by cytoplasmic sequestration of BAD that prevents BCL2 activation and subsequently caspase activation. Thus a reduced phosphorylation of BAD in diabetic pancreas is consistent with increased apoptosis [54]. The inventors' data also showed increased apoptosis in the diabetic mouse pancreas measured by the increased number of β-cells expressing caspase 3 cleaved. Accordingly, diabetic mice rescued by hBMSC-VEGF showed a significant reduction in apoptosis.

In keeping with the observation of enhanced insulin/PI-3K/AKT signaling, the pancreatic islets of the rescued mice showed greater expression and nuclear localization of PDX1 compared with diabetic mice. PDX1 is a downstream transcriptional target of insulin signaling and it is required for β-cell growth and differentiation [27]. Moreover the expression level of p27^{Kip1}, a cell cycle inhibitor known to be negatively regulated by PI-3K/AKT pathway in β-cells through FoxO1[31] and Gsk-3β[30], was significantly reduced in the rescued group while up-regulated in control and diabetic pancreatic islets. This finding confirmed the increased proliferative signal in the rescued pancreatic islets through the Insulin receptor/PI-3K/ATK pathway, compared with both control healthy mice and diabetic mice.

In addition, PI-3K signaling is also known to modulate VEGF expression in the endothelial cells and to induce angiogenesis [29] though v-Src [55]. Moreover, VEGF expression is predominant in the pancreatic β-cells and VEGF receptor 2 (VEGFR2) is highly expressed in the intra-islets capillary [20]. The inventors' data showed a dramatic increase in VEGF expression in the β-cells of rescued mice compared with diabetic mice, implying the possible activation of VEGF expression via PI-3K/AKT pathway.

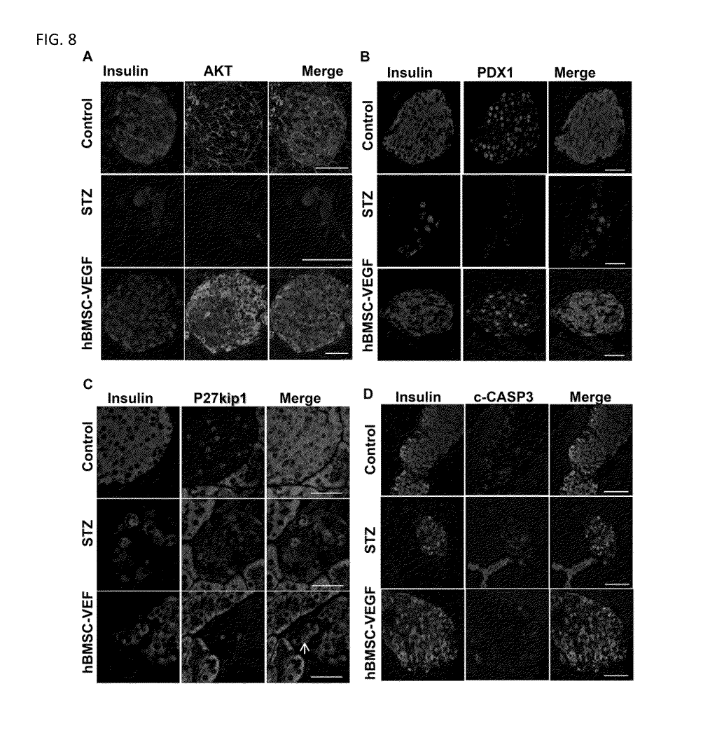

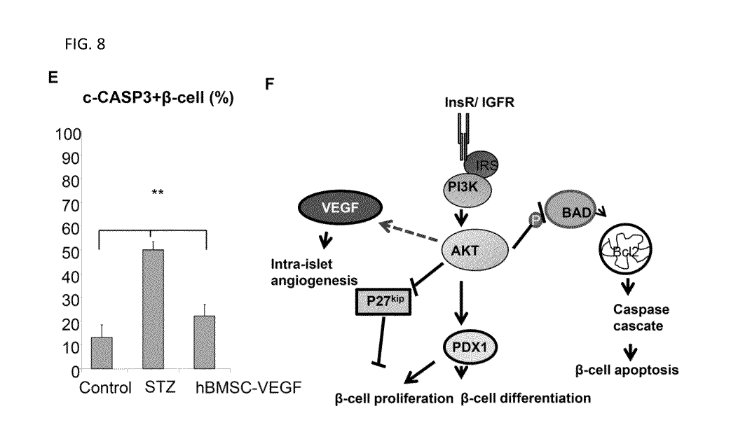

Taking together, the inventors' results suggest that hBMSCs-VEGF induce reversion of diabetes mainly by induction of endogenous β-cell regeneration through the generation of a favorable microenvironment and through the activation of the insulin/IGF1/PI-3K/AKT pathway. Activation of this pathway in the β-cells improves cell survival through inhibition of apoptosis and induces β-cell differentiation and proliferation through activation of PDX1 expression and inhibition of P27^{Kip1}. In addition, the inventors provide evidence of a possible new mechanism of β-cell recovery/regeneration through modulation of intra-islet angiogenesis. The activation of the insulin/IGF signaling through the PI-3K pathway in the diabetic mice rescued by hBMSC-VEGF induces VEGF expression in the β-cells and correlated with β-cell recovery (FIG. 8F).

In conclusion, the inventors' work provides new insight into the mechanism of β-cell recovery after injury mediated by hBMSC therapy and demonstrates the th ability to use hBMSC expressing VEGF for the treatment of insulin-dependent diabetes.

Embodiments of the present invention are based, at least in part, by these findings.

Various embodiments of the present invention provide for a method of producing pancreatic precursor cells from whole bone marrow and insulin-producing cells from whole bone marrow. The method can comprise chromatin remodeling, induction, differentiation and maturation.

Chromatin remodeling can comprise providing nestin-positive bone marrow-derived neurospheres, culturing the nestin-positive bone marrow-derived neurospheres in a first culture medium (basal medium); contacting the nestin-positive bone marrow-derived neurospheres with 5-AZA; changing the first culture medium; and contacting the cells with TSA. In various embodiments, the first culture medium can comprise a component selected from the group consisting of KO-DMEM, β-mercaptoethanol, L-glutamine, non-essential amino acid, BSA, N2 supplement, B27 supplement, heparin, bFGF, EGF, and combinations thereof.

Induction can comprise changing the first medium to a second medium (induction medium), culturing the cells for about 7 days. In various embodiments, the second culture medium can comprise one or more components from the first culture medium and a component selected from the group consisting of DMEM, low glucose, ITS, RA and combinations thereof.

Differentiation can comprise culturing the cells in a third medium (differentiation medium) for about 7 days. In various embodiments, the third medium can comprise one or more components from the first culture medium and a component selected from the group consisting of DMEM, high glucose and combinations thereof.

Maturation can comprise culturing the cells in a forth medium (maturation medium) for about 7 days. In various embodiments, the fourth medium can comprise one or more components from the first culture medium and a component selected from the group consisting of DMEM, high glucose, nicotinamide, and combinations thereof. In various embodiments, the forth medium does not contain bFGF and EGF.

In various embodiments, the first, second, third and fourth media are changed about every 2 days within each step.

In various embodiments, β-islet cells and particularly, insulin producing β-cells are produced by these methods.

Various embodiments of the present invention provide for pancreatic precursor cells derived from whole bone marrow and insulin-producing cells from whole bone marrow. In various embodiments the pancreatic precursor cells are produced by methods of the present invention. Other embodiments of the present invention provide for β-islet cells and insulin producing β-cells are produced by methods of the present invention.

Various embodiments of the present invention provide for a method of producing VEGF and PDX1 expressing bone marrow stem cells.

The method can comprise providing bone marrow stem cells and transfecting the BMSCs with a virus carrying PDX1, VEGF, or both. In various embodiments, the virus is an adenovirus.

In various embodiments, bone marrow stem cells can be obtained by providing bone marrow aspirate, culturing the nucleated cells from the bone marrow aspirate in a culture medium; separating the non-adherent cells from the adherent cells; washing the adherent cells and culturing the adherent cells; harvesting the cells and expanding the cells. In various embodiments, the culture medium can comprise a component selected from the group consisting of Afla-Men, fetal bovine serum, glutamine, penicillin, streptomycin and combinations thereof.

Various embodiments of the present invention provide for treatment of diabetes and ameliorating symptoms of diabetes.

In various embodiments, the method can comprise, providing insulin-producing β-cells differentiated in vitro from BMSC, BMSC expressing VEGF, BMSC expressing PDX1, or BMSC expressing both VEGF and PDX1, and administering the BMSC, the BMSC expressing VEGF, PDX1 or both to a subject in need thereof. In various embodiments, the BMSC expressing VEGF and/or PDX1 are BMSCs that are artificially induced to express VEGF and/or PDX1; for example, by transfection of an adenovirus vector expressing VEGF and/or PDX1.

In various embodiments, the method can comprise obtaining bone marrow from a subject in need thereof, producing insulin-producing β-cells from the bone marrow, and transplanting the insulin-producing β-cells back into the same subject. In various embodiments, transplantation could be intra-hepatic or subcutaneous. In various embodiments, producing insulin-producing beta-cells from the bone marrow may be through the methods of the present invention.

In various embodiments, the method can comprise isolating BMSC from a bone marrow sample from a subject; genetically modifying the BMSC to increase or induce expression of VEGF and/or PDX1, and transplantation of BMSC into the circulation of the same subject by intravenous injection. In various embodiments, the BMSCs are undifferentiated. Methods of genetically modifying the BMSCs to increase or induce expression of a gene in a cell are known in the art and can readily be used in the present invention.

In various embodiments, the present invention provides pharmaceutical compositions including a pharmaceutically acceptable excipient along with a therapeutically effective amount of pancreatic precursor cells or insulin-producing cells derived from whole bone marrow. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Typical dosages of an effective quantity of pancreatic precursor cells, insulin-producing cells derived from whole bone marrow, BMSC expressing VEGF and/or PDX-1, or insulin-producing β-cells derived from BMSCs can be as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biopsied malignant tumors, or the responses observed in the appropriate animal models, as previously described.

The present invention is also directed to kits to produce pancreatic precursor cells or insulin-producing cells derived from whole bone marrow, and kits to treat diabetes. The kits are useful for practicing the inventive method of producing pancreatic precursor cells or insulin-producing cells derived from whole bone marrow and treating diabetes. The kit is an assemblage of materials or components, including at least one of the inventive compositions. Thus, in some embodiments the kit contains a composition including pancreatic precursor cells or insulin-producing cells derived from whole bone marrow, as described above.

The exact nature of the components configured in the inventive kit depends on its intended purpose. For example, some embodiments are configured for the purpose of treating diabetes; some embodiments are configured for the purpose of producing pancreatic precursor cells or insulin-producing cells derived from whole bone marrow. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to produce pancreatic precursor cells or insulin-producing cells derived from whole bone marrow, or to treat diabetes. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in cell therapy. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing, whole bone marrow, bone marrow derived neuropheres, pancreatic precursor cells derived from whole bone marrow, insulin-producing cells derived from whole bone marrow, BMSC expressing VEGF and/or PDX-1, or insulin-producing β-cells derived from BMSCs. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Cell Isolation and Culture

Adult Fisher rats were purchased from Charles River Laboratories (Wilmington, Mass., USA). They were killed by CO₂ asphyxiation according to an approved protocol by the Institutional Animal Care and Use Committee at Cedars Sinai Medical Center. Whole bone marrow was harvested from the femurs as previously published (Talmor et al. 1998). Briefly, the femurs were isolated from the muscle tissues and both ends of the bones were cut. The marrow was flushed and the tissue was passed through a mesh to remove small pieces of debris. After washing, nestin-positive spheres were obtained as described previously by the inventors' group (Kabos et al. 2002). Cells were plated at a density of 1×10⁶ cells/well in poly-D lysinecoated 24-well plates (BD Biosciences, San Jose, Calif., USA) in serum-free DMEM/F12 medium (Invitrogen), supplemented with 20% B27 (Invitrogen), 20 ng/ml of fibroblast growth factor (bFGF, Peprotech, Rocky Hill, N.J., USA), 20 ng/ml of epidermal growth factor (EGF, Peprotech), penicillin (100 U/ml) and streptomycin (100 μg/ml). Medium was changed every 2 days. Cells were maintained at 37° C. in a humidified atmosphere containing 5% CO₂. Portions of cells from sphere forming were dissociated to single cells, frozen down (10% dimethylsulfoxide and 40% fetal bovine serum) by cryotank in −80° C. for overnight and put into the liquid nitrogen (LN2) tank for maintenance of cell populations.

For control experiment rat bone marrow mesenchymal stem cells (MSCs-BM) were cultured on a-MEN medium (Invitrogen) supplemented with 10% FCS (Invitrogen). Briefly, the cells collected from bone marrow (as described earlier) were plated into tissue culture-treated dishes of 100 mm in diameter at 37° C. in a humidified atmosphere containing 5% CO₂. After 24 h, floating cells were washed out and only adherent cells were maintained in the culture with medium changed every 3 days. Cells which reached 80-90% confluency in 100 mm dish were passaged to 1:4 and cultured in the same medium.

### Example 2

### Cell Culture and Differentiation

The inventors' group previously described the successful selection, from rat whole bone marrow, of multipotent stem cells expressing nestin (n-BMSC) and forming a spheroid structure with phenotypic characteristics and a genetic signature typical of neuropheres (Kabos et al. 2002).

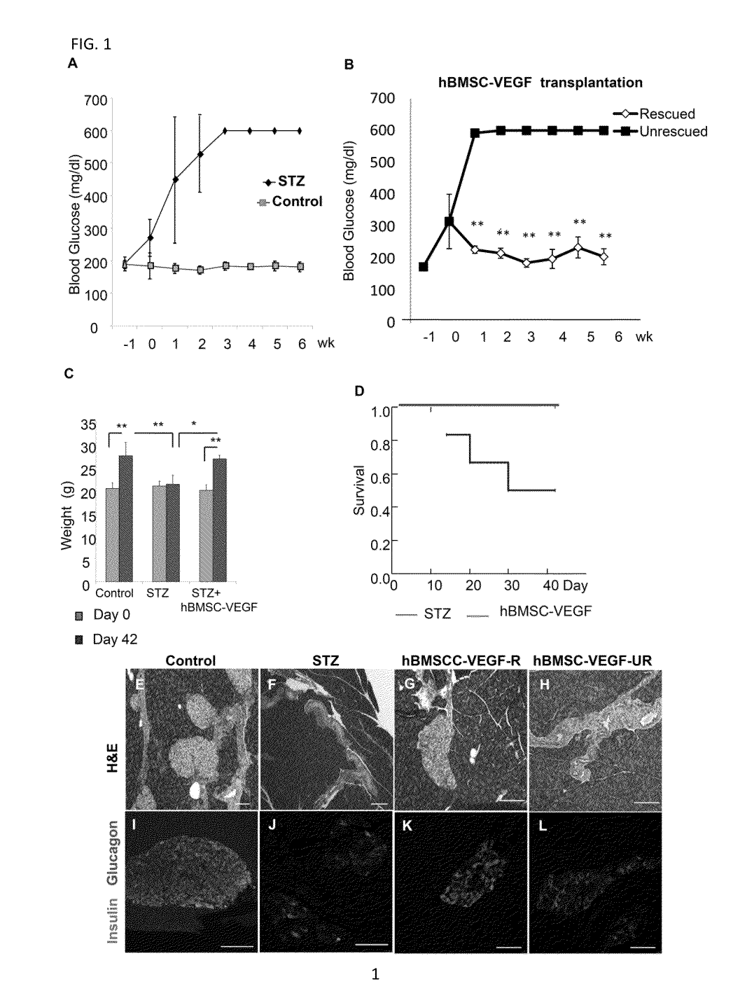

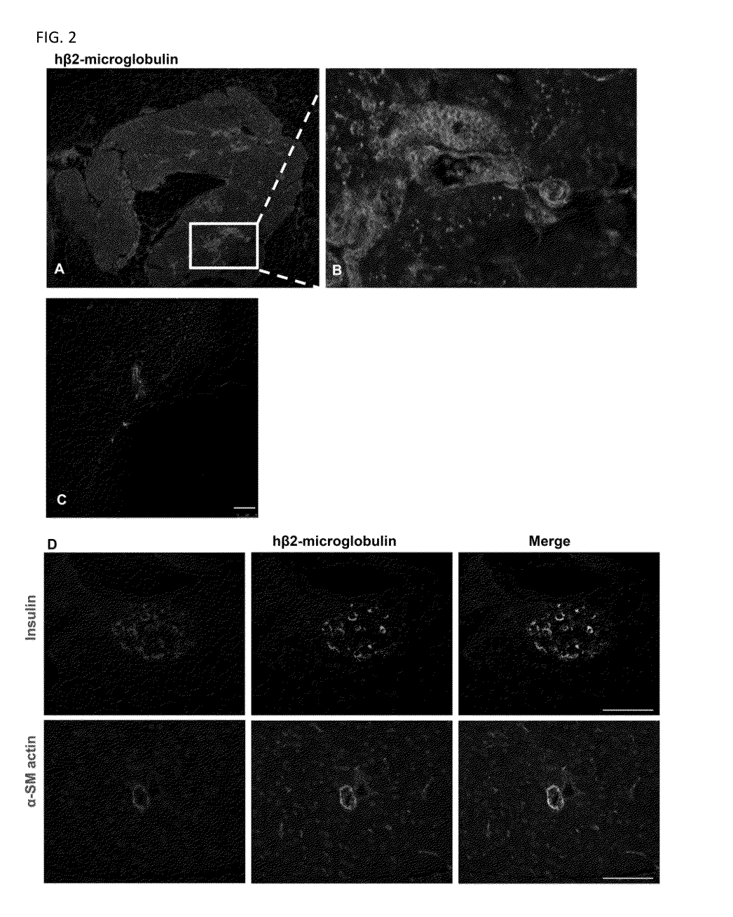

The inventors established a complex differentiation protocol comprising four basic steps (FIG. 1). Control experiments were conducted in parallel using rat MSCs-BM. In the first step (chromatin remodeling), nestin-positive spheres were plated at a concentration of about 100 spheres/ml in each well of 6-well culture plate (Corning, Corning, N.Y., USA), in a basal medium containing KO-DMEM and main components: 0.1 mM β-mercaptoethanol, 2 nM L-glutamine, 1% nonessential amino acid, 0.2% BSA, N2 supplement, B27 supplement (all from Invitrogen), 2 μg/ml heparin, 20 ng/ml bFGF, and 20 ng/ml EGF. The cells were treated with 1 μM 5-AZA (Sigma) for 24 h. After 24 h the medium was changed and cells were treated with 100 nM TSA (Sigma) for 24 h.

In the second step (induction), the basal medium was changed with induction medium containing DMEM with low glucose (1 g/l; Invitrogen), 1× insulin-transferin-selenium (ITS; Invitrogen), 2 μM all-trans RA (Sigma), and main components. The cells were maintained in induction medium for about 7 days. In the third step (differentiation), the cells were seeded into 6-well plates coated with poly-L-ornithine (15 μg/ml; Sigma) at a concentration of 2-5×10⁵ cells/well in differentiation medium containing DMEM with high glucose (Invitrogen) and main components for 7 days. In the last step (maturation) the medium was modified from differentiation medium, adding 10 mM nicotinamide (Sigma) without supplement of bFGF and EGF for 7 days. For each step the medium was changed every 2 days.

### Example 3

### Quantitative Real-Time PCR

Cells were harvested at different steps and total RNA was extracted using RNeasy Minikit (Qiagen), according to the manufacturer's instruction. cDNA was prepared using Superscript reverse transcriptase (Invitrogen). cDNA samples derived from 50 ng of total RNA was analyzed by quantitative reverse transcription-PCR (qRT-PCR) using SYBR green dye with QuantiTect SYBR Green RT-PCR kit (Qiagen).

Sequences of PCR primers used are listed in Table 1. PCR was performed with cycles at 95° C. for 15 s, 56-60° C. for 10 s (Table 1) and 72° C. for 20 s. Reactions (40 cycles) were carried out with iCycler PCR machine (Bio-Rad) and data analysis was performed with QPCR software (Applied Biosystems, Foster City, Calif., USA). Each experiment was performed at least three times. Relative quantitative analysis was performed following 2^{-ΔΔC}_{T}. The expression of each gene was normalized to b-actin gene expression.

TABLE 1
PCR primer sets

| | | SEQ ID | Annealing T |
|---|---|---|---|
| | Primer sequences | NO: | (8C)/time (s) |
| Nestin | F: 5′-gcggggcggtgcgtgactac-3′ | 1 | 58/10 |
| | R: 5′-aggcaagggggaagagaaggatgt-3′ | 2 | |
| Pdx1 | F: 5′-atcactggagcagggaagt-3′ | 3 | 56/10 |
| | R: 5′-gctactacgtttcttatct-3′ | 4 | |
| Ngn3 | F: 5′-ccgcgtggagtgacctctaa-3′ | 5 | 60/10 |
| | R: 5′-ggtggaattggaactgagcactt-3′ | 6 | |
| CK19 | F: 5′-acagccagtacttcaagacc-3′ | 7 | 56/10 |
| | R: 5′-ctgtgtcagcacgcacgtta-3′ | 8 | |
| Insulin | F: 5′-tcttctacacacccatgtccc-3′ | 9 | 55/10 |
| | R: 5′-cacctagtcacgacgtgg-3′ | 10 | |
| Glucagon | F: 5′-acctagactcccgccgtg-3′ | 11 | 55/10 |
| | R: 5′-cttgaacccgcgtctgta-3′ | 12 | |
| PP | F: 5′-cgcatactactgcctctccc-3′ | 13 | 60/10 |
| | R: 5′-cagcagcgcagggcatcaaa-3′ | 14 | |
| Som | F: 5′-ctgcatcgtcctggctttgg-3′ | 15 | 55/10 |
| | R: 5′-tgcagccagctttgcgttcc-3′ | 16 | |
| Glut2 | F: 5′-ggatctgctcacatagtcac-3′ | 17 | 60/10 |
| | R: 5′-ccaagtaggatgtgccagta-3′ | 18 | |
| b-actin | F: 5′-acctgacagactacctcatg-3′ | 19 | 58/10 |
| | R: 5′-atcgtactcctgcttgctga-3′ | 20 | |

PP, pancreatic polypeptide; Som, somatostatin; Glut2, glucose transporter 2.

### Example 4

### Immunocytochemistry

Spheroids were fixed in 4% paraformaldehyde in phosphate buffer for 10 min. Blocking was carried out for 30 min using 10% FCS diluted in PBS at room temperature. The cells were then incubated with primary antibodies in solution of 3% of FCS in PBS for an hour at room temperature. Antibody dilutions were as follows: mouse monoclonal anti-nestin (MAB353) (1:100; Millipore Corporate, Bellerica, Mass., USA), goat anti-PDX1, rabbit anti-PAX6, rabbit anti-CK19, rabbit anti-insulin, goat anti-glucagon, and rabbit anti-amylase (All 1:50, from Santa Cruz Biotechnology, Santa Cruz, Calif., USA). Afterwards, the cells were washed with PBS for 5 min for three times. Secondary antibodies were FITC or Texas red conjugated anti-mouse, anti-goat and anti-rabbit (Vector Laboratories, Inc., Burlingame, Calif., USA) and were diluted 1:500 in PBS containing 3% of FCS. Cells were incubated in this solution for 45 min at room temperature, followed by washes in PBS for 5 min for three times. Matched exposure of control sample was stained using non-immune calf serum in place of the primary antibodies. Nuclear DNA was stained with 4′,6-diamidino-2-phenylindole (Vector Lab). Images were captured by digital camera connected with fluorescent microscope (Model Upright Zeiss, Jena, Germany). Scanning confocal images were obtained by a confocal microscope (Leica Microsystems SP5, Mannheim, Germany).

### Example 5

### Dithizone Staining

Dithizone (DTZ, Sigma), which stains zinc-containing cells bright red, was used to quickly assess the presence of insulin-producing cells. The staining protocol was followed from the study by Shiroi et al. (2002). DTZ stock solution was prepared by solving 50 mg of DTZ in 5 ml of dimethyl sulfoxide (Sigma), sterile-filtering through a 0.22 μm nylon filter, and stored at −20° C. The working solution was prepared (pH 7.8) by diluting the stock solution 1:100 in culture medium. For each dish, 2 ml of DTZ solution was added and incubated for 30 min at 37° C. After washing the cells three times in PBS, the differentiated islet-like clusters were examined under phase contrast inverse microscope.

### Example 6

### Insulin Assay

For each determination, about 100 spheroids of similar size were randomly handpicked at stage 4 (3 weeks after starting the pancreatic differentiation) and incubated in DMEM with low glucose (1 g/l) without serum overnight. For insulin secretion assays spheroids were preincubated for 1 h in Krebs-Ringer/bicarbonate buffer (KRB: 120 mM NaCl, 5 mM KCl, 2.5 mM CaCl₂, 1.1 mM MgCl₂, and 25 mM NaHCO₃) at 37° C. or on ice (for control experiments) in 5% CO₂ atmosphere, followed by incubation for an additional hour in KRB containing 0.5 mM 1-isobutyl-3-methylxanthine and glucose at different concentrations (5, 10, and 20 mM). The buffer was collected and frozen at −70° C. until assay for insulin content. The amount of insulin released was determined by ultrasensitive rat insulin ELISA (Linco Research, Billerica, Mass., USA) according to the manufacturer's protocol. Values showed the mean of three replicates ±S.D.

### Example 7

### Statistical Analysis

Results are shown as mean±S.D. Student t-test was used to compare between two groups. One-way ANOVA was used to compare among three groups. In all statistical analyses, a P value of <0.05 was judged statistically significant.

### Example 8

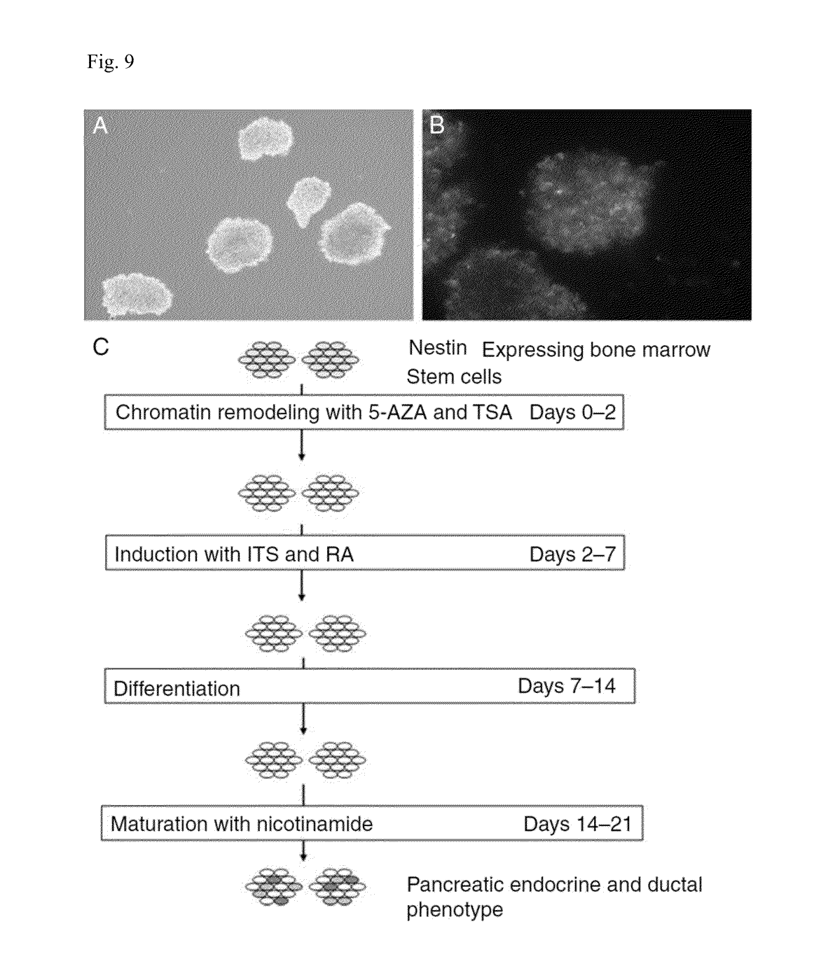

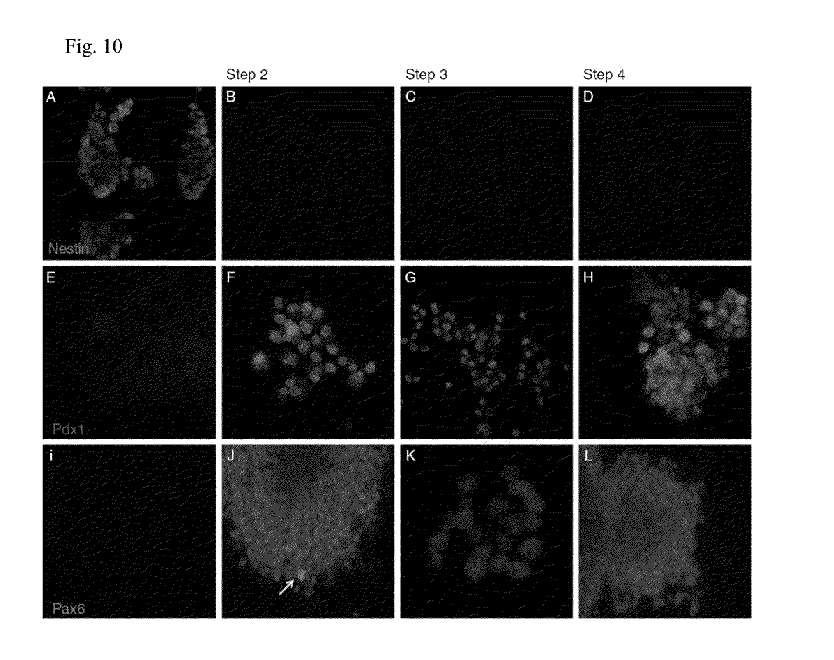

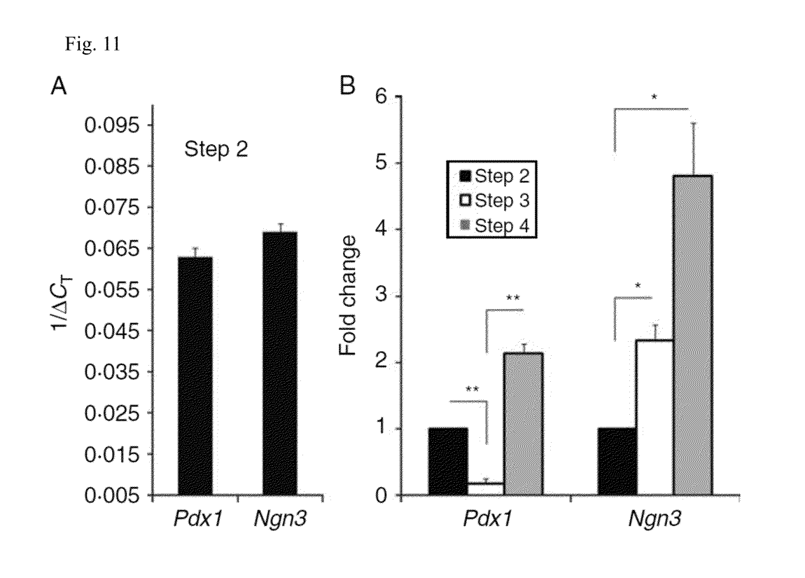

To drive differentiation of bone marrow stem cells expressing nestin towards pancreatic lineages, the inventors developed a culture procedure comprising four steps (FIG. 9). In the first step the cells were exposed to 5-AZA for 24 h and TSA for another 24 h. The dose of 5-AZA used in this study has been successfully used by Lefebvre et al. (2009) to induce NGN3 expression and endocrine differentiation into the PANC-1 human ductal cell line. The inventors determined the optimal concentrations of TSA by testing cell survival, growth and death with increasing doses from 10 nM to 1 mM. Concentrations of more than 100 nM for TSA showed an increased cell death and reduced differentiation capability (data not shown). The inventors rationalized that chromatin remodeling is the first step to induce nestin-positive cells because of putative cell lineage differences and specification, and believed that the increased ‘susceptibility’ of chromatin by serial epigenetic modifications will enhance the induction and further direct differentiation toward the pancreatic phenotype (FIG. 9). After exposure to TSA, the cells were cultured for 7 days in a serum-free medium containing ITS and RA (FIG. 9C, induction). The concentration of RA adopted in this study was previously optimized in embryonic stem cell differentiation model (Cai et al. 2009). Recently, the role of RA in pancreatic development and early endocrine lineage determination has been shown (Ostrom et al. 2008). In addition, RA has been used for both mouse and human embryonic stem cells to induce differentiation into pancreatic cell lineage (Micallef et al. 2005, Johannesson et al. 2009). At this time point the inventors were able to highly induce the expression of a pancreatic master gene (pancreas duodenum homeobox-1, PDX1), together with other genes important in the early pancreatic development (PAX6 and NGN3). Many cells were positive for PDX1 as assayed by immunohistochemistry (FIG. 10F). Subsequently, the PDX1-positive cell population dramatically decreased after 7 days during the third step (differentiation; FIG. 10G) but increased again and became prominent in the fourth step (maturation; FIG. 10H). This fluctuation of PDX1-positive cell population by immunohistochemistry also appeared in the qRT-PCR analysis in sequential manner (FIG. 11B). Pdx1 gene expression was initiated within 7 days during the second step culture (induction), then downregulated several fold but was detectable ˜2 weeks later during the third step, and upregulated again 3 weeks later during the maturation step. Ngn3 transcript was induced at day 7 and progressively increased two- to fivefold at day 14 (step 3) and day 21 (step 4) respectively (FIG. 11). Only a few cells were positive for PAX6 at day 9, as confirmed by immunohistochemistry (FIG. 10) and the number of positive cells strongly increased during step 3, but was not detectable at step 4. Surprisingly, these results were consistent with the physiological expression of PDX1, PAX6, and NGN3 genes during in vivo pancreas development. In contrast, nestin expression was high in the beginning of the differentiation and dramatically decreased to an undetectable level during the second stage of conditioning. This was confirmed by both immunohistochemistry and (FIG. 10A-D) and qRT-PCR (Table 2). Morphologically, the cells stayed aggregated in spheroid structure.

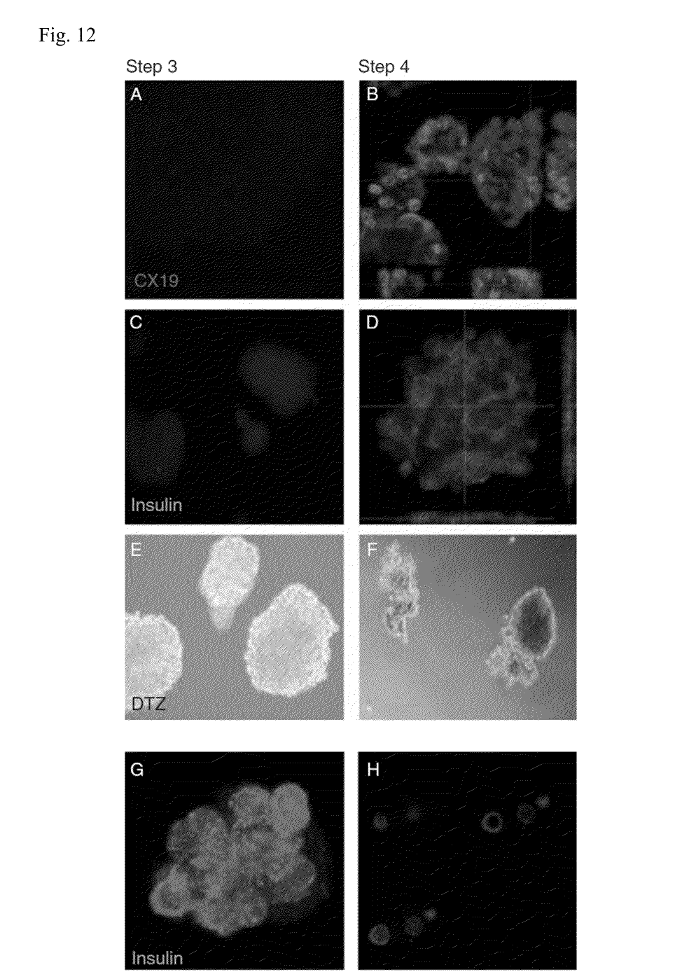

In the third stage of conditioning the inventors used poly-Lornithine-coated dishes with a medium containing high glucose with both bFGF and EGF. In such an environment, the cells underwent further differentiation into a pancreatic phenotype. At about 2 weeks of the differentiation process, corresponding to the maximized expression of PAX6, increased expression of NGN3 and downregulation of PDX1, endocrine markers for a more mature phenotype started to appear as confirmed by qRT-PCR: insulin, glucagon, and glucose transporter 2 (Glut2; Table 2). The expression of Ck19, a ductal marker, was also induced. At this stage immunohistochemistry was negative for the same markers (FIG. 12).

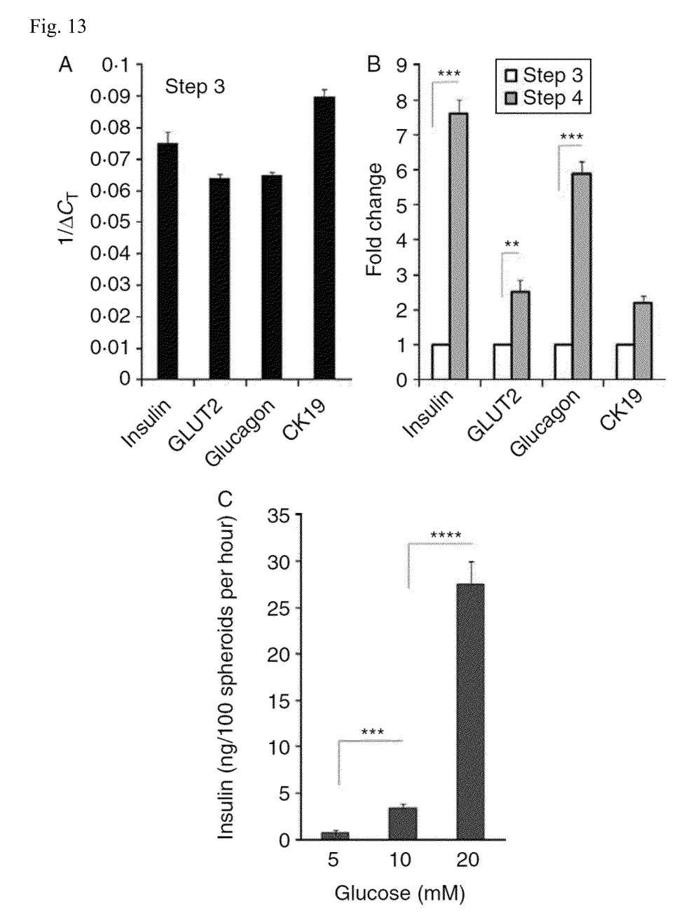

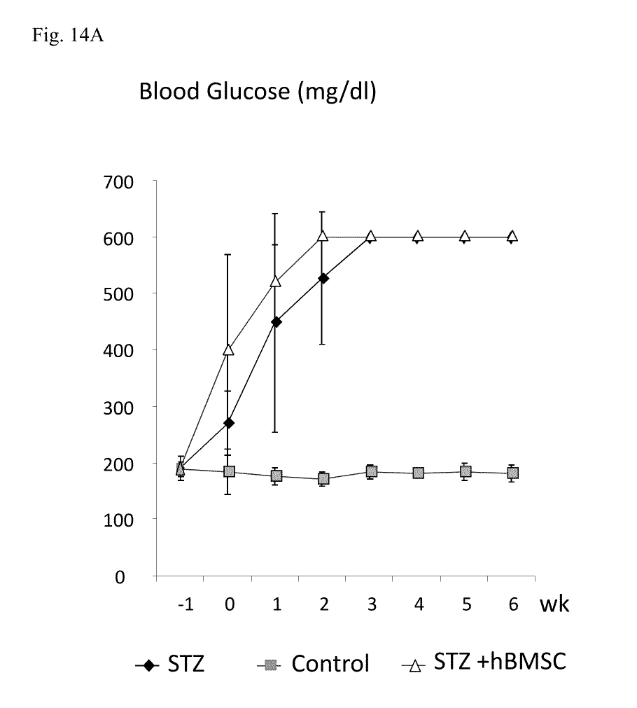

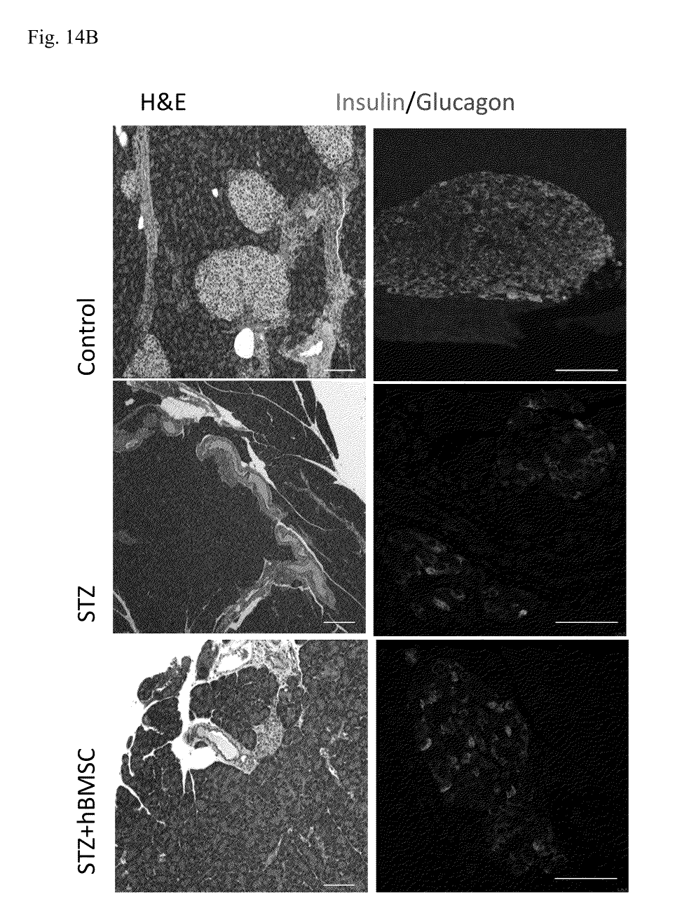

In the last step, the inventors tried to further push the differentiation toward a more mature phenotype. For this reason the inventors used nicotinamide for about 7 days. At day 21 from the beginning of the in vitro differentiation, a mixed population of cells expressing endocrine and ductal phenotypes were observed. Immunofluorescent staining was markedly positive for insulin and CK19 (FIG. 12). These cells also positively stained with the zinc chelator, DTZ, indicating the presence of intracellular proinsulin, as has been previously observed for ‘pseudoislets’ in culture (Kuo et al. 1992). Only a small number of cells were positive for glucagon with immunofluorescent staining (data not shown). Insulin, CK19, and glucagon transcripts were also consistently upregulated (seven- to eightfold, five- to sixfold and two- to threefold, respectively; FIGS. 13A and 13B). No amylase was detected on immunohistochemistry (data not shown) and RNA levels (Table 2), indicating the absence of a pancreatic exocrine phenotype. In this culture condition the inventors also failed to detect somatostatin and pancreatic polypeptide-positive cells (Table 2).

TABLE 2
Expression of pancreatic transcription factors and islet hormone
transcripts at different time points during in vitro differentiation

| | | Step 2 | Step 3 | Step 4 |
|---|---|---|---|---|
| | Time 0 | (day 9) | (day 16) | (day 23) |
| Nestin | 26 ± 1 | — | — | — |
| Pdx1 | — | 29 ± 0.5 | 32 ± 1 | 27 ± 1 |
| Ngn3 | — | 28 ± 0.2 | 27 ± 0.2 | 26 ± 0.1 |
| Insulin | — | — | 27 ± 0.2 | 23 ± 1 |
| Glucagon | — | — | 29 ± 0.1 | 26 ± 0.2 |
| Glut2 | — | — | 29 ± 0.1 | 27 ± 0.5 |
| Ck19 | — | — | 25 ± 0.1 | 23 ± 0.1 |
| Amylase | — | — | — | — |
| Somatostatin | — | — | — | — |
| PP | — | — | — | — |

RNA from n-BMSC was extracted at different time points during in vitro differentiation and analyzed by quantitative reverse transcription-PCR for transcripts encoding pancreatic transcription factors and the four islet hormones. Values represent cycle threshold±S.D. PP, pancreatic polypeptide. hormones. Values represent cycle threshold±S.D. PP, pancreatic polypeptide.

To evaluate the functionality of differentiated cells, the inventors tested for glucose-induced insulin secretion in vitro. At the end of the last differentiation stage, the cell aggregate (spheroids) not only produced insulin, but also displayed a glucoseresponsive secretion of the hormone. Insulin secretion was dose-dependent after incubation for 1 h with glucose (5, 10, and 20 mM; FIG. 13C). Spheroids incubated on ice under the same condition did not show glucose-responsive insulin secretion. The average insulin secretion on ice with glucose 5, 10, and 20 mM was 0.85±0.32, 1.5±0.41, and 1.8±0.5 ng/100 spheroids per hour respectively. This is consistent with inhibition of glucose-induced insulin release by cooling (Atwater et al. 1984). These findings demonstrate de novo synthesis and processing of insulin and physiologically regulated secretion. Moreover the cells expressed a functional element, Glut2 (FIGS. 13A and 13B) that allows the rapid entry of glucose into the cells. This confirmed the capability of the inventors' differentiated cells to respond to glucose stimulation.

### Example 9

### Human BMSC Culture and Expansion

Bone marrow aspirate from normal adult donor was purchased from Allcell (Emeryville, Calif.). All of the nucleated cells were plated in 100-cm² culture dish (Corning, Corning, N.Y.) in a basal medium consisting of Alfa-Men, 17% fetal bovine serum, 2 mM glutamine, 50 U/L penicillin and 25 μg/L streptomycin (all from Invitrogen). After 24 hr in culture, non-adherent cells were separated from adherent cells. The adherent cells were washed with PBS and cultured with the previous medium for 5-7 days. At 80% confluences the cells were harvested with trypsin/EDTA and plated in two 100-cm² dishes (passage 1). Cell underwent two further expansions before aliquots were storage in 90% FBS and 10% DMSO and frozen in −80° C. Until passage 6-7 the cells were growing with doubling time of 48 hrs. All the experiments were performed using a single batch of hBMSCs from a single donor. The cells were used within passage 7. Human BMSCs from passage #3 to 6 were analyzed for various cell surface markers commonly used for the positive and negative detection of mesenchymal stem cells by flow cytometry analysis (CD44, CD31, CD34, and CD 105). To characterize the mesenchymal potential of hBMSCs, the inventors induced adipogenic and osteogenic differentiations in vitro (data not shown).

### Example 10

### Flow Cytometric Analysis

Human BMSCs were analyzed for surface expression of different markers commonly used for the positive and negative detection of mesenchymal stem cells from passage 3 to 6. Cells were detached with tripsin/EDTA and incubated with respective fluorochrome-conjugated antibodies for cell surface glycoproteins: CD44, CD31, CD34, and CD 105 (BD Biosciences, San Diego). Fluorochrome-conjugated Isotype IgG were used as control. Cells were immediately analyzed by BD FACScan and analyses were done using BD CellQuest software (BD Biosciences), and percent statistics were given.

### Example 11

### Adenovirus Production and Cell Infection

c-DNAs encoding for human Pdx1 and mouse VEGF165 were subcloned into Adeno-X viral DNA vector (BD Biosciences Clontech), following manufacturing protocol #PT3414-1, Version #PR31147. CMV has been used as promoter. Successful homologous recombination resulted in recombinant virus encoding for PDX1 (Ad-PDX1) and VEGF (Ad-VEGF). The virus was expanded in HEK293 cells as described in the ViraPower Adenoviral Expression system manual by Invitrogen. The viral titer was determinate using a kit from Clontech and found a pfu of ˜1.1×10 Ê9 for all 2 constructs. Human BMSCs were transfected with adenovirus carrying PDX1 (hMSC-BM-Pdx1) or carrying VEGF (hMSC-BM-VEGF) 2 days before transplantation. RNA and protein level of PDX1 and VEGF in the transfected cells were assessed by PCR and Western Blotting.

### Example 12

### In Vivo Animal Model and Stem Cell Transplantation

To induce diabetes, NOD/scid mice (The Jackson Laboratory, Bar Harbor, Me.) 6-8 weeks of age were given three intraperitoneal injections of streptozotocin (STZ) [2-Deoxy-2-(3-methyl 1-3-nitrosoureido)-D-glucopyranose, STZ; Sigma-Aldrich, Saint Louis, Mo.], 50 mg/kg, on day 1-3. All experiments and procedures were performed according to an approved protocol by the Institutional Animal Care and Use Committee at Cedars Sinai Medical Center. STZ was dissolved in sodium citrate buffer, pH 4.5, and injected within 15 minutes into fasted mice. A total of 36 mice were used for this study. One control group (n=4 mice) did not received any treatment, another group (n=6) received a sham injection after induction of diabetes with STZ. The other 3 groups received stem cell transplantation after induction of diabetes, hBMSC (n=6), h-BMSC-Pdx1 (n=8), or h-BMSC-Vegf (n=9) respectively. Additionally, two groups of STZ treated mice were transplanted with mouse fibroblasts transfected with adenovirus expressing PDX1 or VEGF.

On day 0, about 7 days from STZ treatment, mice were transplanted with about 1×10⁶ cells each. To avoid aggregation of the cells, hBMSC were suspended in 150 μl and injected though the chest wall into the left cardiac ventricle as previously described [7], using a 30 gauge needle. Cells were infected with adenovirus vector 2 days prior to injection into recipient mice. Mouse skin fibroblasts were used as control and transfected with adenovirus expressing PDX1 and VEGF and transplanted in similar conditions (4 mice for each group). All the animals were sacrificed at 6 weeks after stem cell transplantation and peripheral blood, and tissues were collected. Achievement of normoglycemia was defined as blood glucose <200 mg/d1.

### Example 13

### Blood Glucose and Serum Insulin Measurements

Blood glucose was measured in non-fasten mice between 9 and 11 am two times a week. The level of glucose was measured from the tail vein using One Touch Ultra Meter and Test Strips (Lifescan Inc., Milpitas, Calif.). The sensitivity of the assay does not exceed 600 mg/dl, so the maximal extent of hyperglycemia maybe greater than indicated. Mouse serum insulin was determinate by ultrasensitive mouse insulin enzyme-linked immunoabsorbent assay (ELISA) (Alpco Diagnostics, Salem, N.H.) and human serum insulin level by human insulin ELISA (Linco Research, Millipore Corporation, Billerica, Mass.) according to the manufacturer's protocol at 6 weeks after stem cell injection in fasten animals. Three replicates for each sample were used.

### Example 14

### Immunohistochemical Analyses

The pancreatic tissues obtained from mice were harvest 6 weeks after stem cell injection and immediately fixed with 4% paraformaldehyde at 4° C. overnight. The tissues were then dehydrated in graded ethanol, cleared in xylene and finally embedded in paraffin (Paraplast, Leica Surgipath). Blocks were cut at 5 μm on a rotary microtome (LEICA®). For immunohistochemical staining of the paraffin embedded samples, sections were deparaffinized in xylene and rehydrated through ethanol baths and PBS, followed by rinsing in distilled water for 5 min. Pancreatic sections were stained in Harris hematoxylin solution (Sigma) and eosin Y solution (Sigma). For immunofluorescent staining, antigen retrieval was performed by heating at 90° C. in antigen retrieval buffer (DAKO). Pancreatic islets were stained with various primary antibodies: mouse monoclonal anti glucagon (1:100, Sigma-Aldrich), mouse monoclonal anti VEGF (1:100, Novus Biological), rabbit polyclonal anti insulin (Santa Cruz, dilution 1:100), rabbit polyclonal anti-p27Kip1 (Abcam, dilution 1:200), goat polyclonal anti-PDX1 (Santa Cruz, dilution 1:100), mouse monoclonal anti-AKT (Cell Signaling, dilution 1:100), and rabbit polyclonal anti-caspase 3 cleaved (Cell Signaling, dilution 1:100). To evaluate engraftment of human stem cells in the mouse pancreas the inventors used rabbit anti-human β2-microglobulin antibody at a dilution of 1:100 (Abcam). Pancreatic section were blocking on 10% donkey serum (Sigma). To evaluate engraftment of human stem cells in the mouse pancreas the inventors used rat human β2-microglobulin antibody at a dilution of 1:100 (Abcam). Negative controls were incubated with rabbit IgG diluted to 1:100. After washing in PBS, detection of bound primary antibody was carried out with Alexa Fluor 488 or 568 secondary antibodies donkey anti rabbit (Invitrogen). For the double staining experiments to identify possible differentiation of human cells in the mouse pancreas, the inventors used a multiple steps protocol. The slides were incubated first with the following antibodies: mouse monoclonal anti insulin (dilution 1:200), mouse monoclonal anti-α-smooth muscle cell actin (dilution 1:100) (both antibodies cross-react with mouse and human insulin), followed by secondary antibodies Alexa Fluor 568 donkey anti mouse (Invitrogen) at dilution 1:200, and Texas red conjugated rabbit anti goat (Jackson ImmunoResearch, dilution 1:500). After washing in PBS, the slides were incubated overnight at 4° C. with human β2-microglobulin antibody, followed by Alexa Fluor 488 secondary antibody. Nuclear DNA was stained with 4′,6-diamidino-2-phenylindole (DAPI, Vector Lab).

### Example 15

### β Cells Count

Briefly, the pancreatic sections were sequentially incubated with anti-insulin/proinsulin mouse monoclonal antibody (Abcam; dilution 1:200), biotinylated rabbit anti-guinea pig antibody (1:200 dilution; Santa Cruz), and streptavidin-alkaline phosphatase complex (Santa Cruz), for a period of 45 min each. The alkaline phosphatase activity was identified using new fuchsin under light microscope. The sections were counterstained with Harris hematoxylin. The images of the above stained sections were captured at 100× magnifications. The inventors selected 3 sections separated from 200 μm and counted the number of insulin expressing cells in all 3 sections. The inventors used immunofluorescent staining and confocal imaging to quantify β-cells expressing VEGF, human β2-microglobulin, and caspase 3 cleaved. The inventors used pancreas from 3 mice from each group.

### Example 16

### Phase Contrast and Confocal Microscopy Analysis

Images were captured by digital camera connected with fluorescent microscope (Model Upright Zeiss). Scanning confocal images for immunofluorescence analysis were obtained by a laser scanning confocal microscope (Leica Microsystems SP5, Mannheim, Germany).

### Example 17A

### Real-Time PCR Arrays

Pancreatic tissues were preserved with RNA Later (Invitrogen) at −20° C. Total RNA was extracted using RNeasy Minikit (Quiagen), according to the manufacturer's instruction. mRNA from healthy control mice, STZ diabetic mice and STZ diabetic mice rescued by hBMSC-VEGF were obtained as previously described for the Real Time PCR. Briefly, RT2 First Strand Kit (SABiosciences, Frederick, USA) was used to convert mRNA to cDNA. This cDNA was then added to the SAbiosciences RT2 SYBR Green qPCR Master Mix. Each sample was used to performed quantitative gene expression analysis on specific arrays for the insulin signaling pathway (Cat#PAMM-030-F). All steps were done according to the manufacturer's protocol for the Roche Light Cycler 480. The online tool (http://www.sabiosciences.com/pcrarraydataanalysis.php (last accessed Aug. 1, 2011) offered by the manufacturer was used to analyze the data, including significant value and fold changes. Each set of data was repeated 4 times. Only significant results (p<0.05) were taken into consideration.

### Example 17B

### DNA Extraction and Real-Time PCR

Frozen tissues were homogenized, and genomic DNA was extracted using DNeasy Blood & Tissue Kit (Qiagen) from mouse organs and human BMSCs. Mouse DNA was isolated from identical tissues of non-transplanted NOD/SCID mice as used as negative control. In addition, human DNA was isolated from hBMSC cultures and used as positive control. Total DNA was assayed by UV absorbance. Real time-PCR was performed with 100 ng target DNA. To detect human DNA in the mouse tissues the inventors used previously reported human specific primers, targeting a unique and conserved region of human β-actin [26]. Endogenous mouse GAPDH gene (Qiagen, QT01658692) was also amplified as internal control. Real-Time PCR was carried out with iCycler (Bio-Rad), using QuantiFast SYBR Green PCR kit (Qiagen), following manufacture protocol. Absolute standard curves were obtained for the human β-actin and mouse GAPDH. To evaluate human specificity of human β-actin gene, standard curves were generated by serially diluting human genomic DNA in mouse DNA. Values are expressed in percent of human DNA infused as cells in the mouse tissue. Each assay was carried on in triplicates and repeated at least 3 times.

### Example 18

### Statistical Analysis

All data were presented as mean±SD and were compared by student T-test. P<0.05 was considered to indicate statistical significance of the test results. Kaplan-Meier curves were used for the survival study and the log-rank (Maltel-Cox) test was used to determine statistical significance. For multiple comparisons the inventors used one way ANOVA followed by Tukey test.

### Example 19

### Human Bone Marrow Stem Cells Did not Induced Recovery of STZ-Induced Diabetes

Human stem cell isolated from bone marrow (hBMSC) did not express hematopoietic markers such as CD34, endothelial markers such as CD31 and most cells at different passage of culture expressed the typical mesenchymal stem cell marker (CD 105, CD 44).

The inventors' hBMSCs expressed mesenchymal cell markers and differentiated into adipogenic and osteogenic cell lineages in vitro (data not shown), confirming their multipotent nature.

Diabetes was induced in NOD/SCID mice with streptozotocin (STZ), a cytotoxic agent that preferentially damages β-cells. All mice treated with STZ (n=6) developed hyperglycemia 6-7 days after STZ injection and 50% of them died before 6 weeks (FIG. 1A). Control untreated mice (n=5) maintained euglycemia during the study period (FIG. 1A). The inventors tested the ability of hBMSCs genetically modified to transiently express VEGF gene to rescue diabetic mice. These cells were injected into the circulation of the STZ-induced diabetic mice (n=9) one week after STZ treatment. Reversion of hyperglycemia was observed in 5 of 9 mice treated with hBMSC-VEGF between 1 to 2 weeks after cell injection and near-normoglycemia remission was maintained for 6 weeks (FIG. 1B, ‘rescued’). Four of the mice treated with hBMSC-VEGF failed to reverse hyperglycemia (‘unrescued’).

Although divided in two groups, rescued and unrescued, all mice treated with hBMSC-VEGF showed better clinical outcomes in terms of survival rates and weight gain compared with the diabetic STZ treated mice (FIG. 1C,D). All of rescued and unrescued groups of mice gained weight significantly, which is comparable to the healthy control mice (FIG. 1C). These mice survived by the end time of the study in contrast to the high mortality rate of the diabetic mice (FIG. 1D, p<0.05). Overall, the response to treatment in terms of reversion of hyperglycemia was significantly higher in the mice treated with hBMSCs-VEGF compared with the mice receiving sham injection (p=0.025).

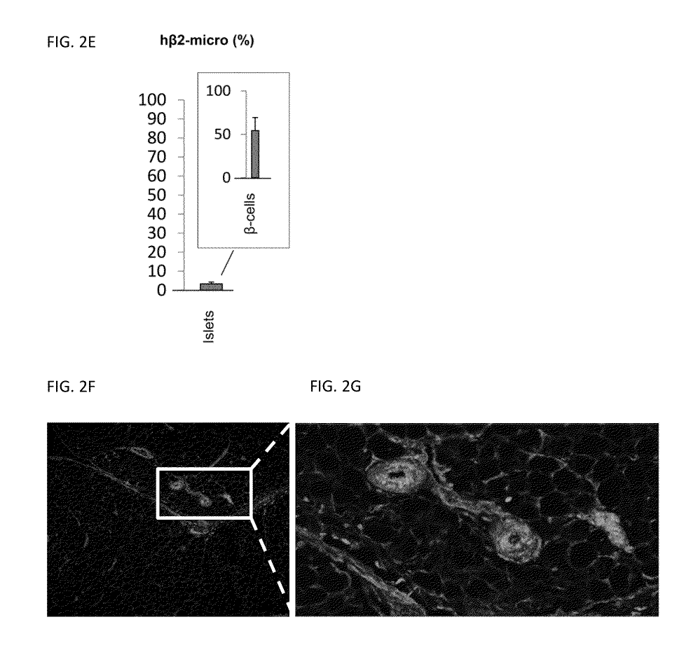

Histological examination at 6 weeks from transplantation showed severe alteration of the pancreatic islet morphology and significant reduction of the number of insulin-expressing cells in the STZ-induced diabetic mice (FIG. 1F,J) and in the unrescued mice (FIG. 1H,L). In contrast, the morphology of pancreatic islet was maintained and the staining patterns of insulin in the pancreatic islets of hBMSCs-VEGF treated mice (FIG. 1G,K) were very similar to one in the healthy control mice (FIG. 1E,I). Further investigation showed that hBMSCs-VEGF were robustly engrafted and diffusely survived in the pancreas (FIG. 2A-B), while fewer human cells were present in the pancreas of the unrescued animals at 6 weeks after transplantation (FIG. 2C). Engraftment and survival of hBMSCs-VEGF in the mouse pancreas was also assessed by real-time PCR for a human-specific gene (Table 1). The pancreatic samples from mice treated with hBMSC-VEGF showed variable amount of human DNA. Small amount of DNA was also present in the kidney of few mice but not in other organs (Table 1).

Furthermore hBMSCs-VEGF were able to differentiate into vessels and β-cells as confirmed by co-staining with human β2-microglobulin which specifically stained human cells and either a-smooth muscle actin or insulin (FIG. 2D). Vascular differentiation was prominent inside the pancreas, however, the efficiency of differentiation into β-cells was low. It is interesting to note that only a small percentage of pancreatic islets were positive for human β2-microglobulin (FIG. 2E). However, those pancreatic islets containing human cells showed approximately half of the β-cells originated from human (FIG. 2E, right panel).

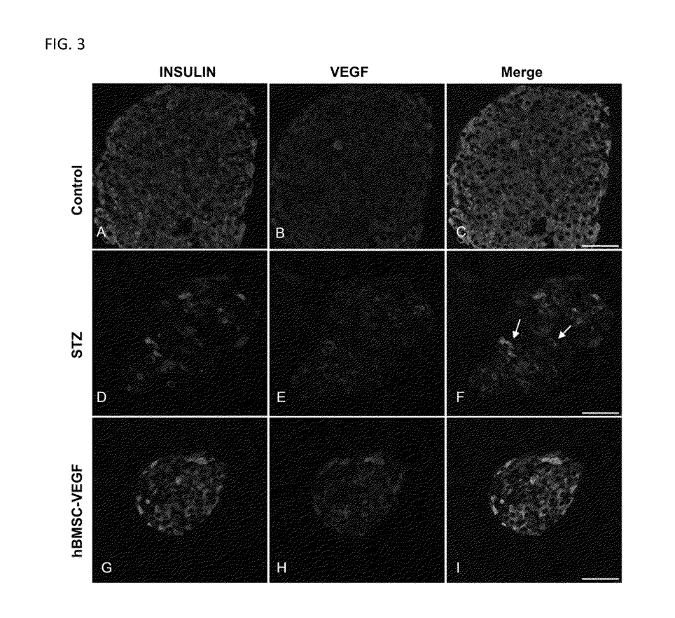

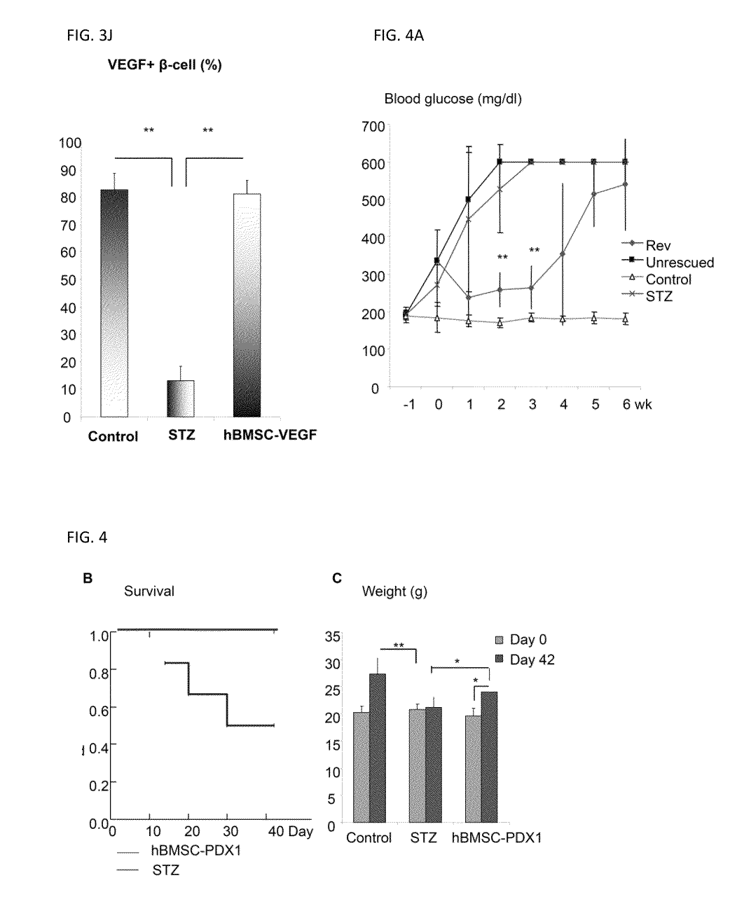

In the healthy age matched control mice, VEGF was uniquely expressed only in the pancreatic isles, mostly by the β-cells (FIG. 3A-C). After induction of diabetes with STZ, the inventors observed a dramatic and significant reduction of VEGF expression in the β-cells (FIG. 3D-F), which was completely restored after treatment with hBMSC-VEGF (FIG. 3A-J).

Further investigation showed that hBMSC-VEGF robustly engrafted and diffusely survived in the mouse pancreas and the mesenchymal tissue surrounding the pancreas of the rescued mice (FIG. 2F-G), while fewer human cells were present in the pancreas of the unrescued animals at 6 weeks after transplantation.

### Example 20A

### Human Bone Marrow Mesenchymal Stem Cell Expressing PDX1 Transiently Ameliorates STZ-Induced Hyperglycemia

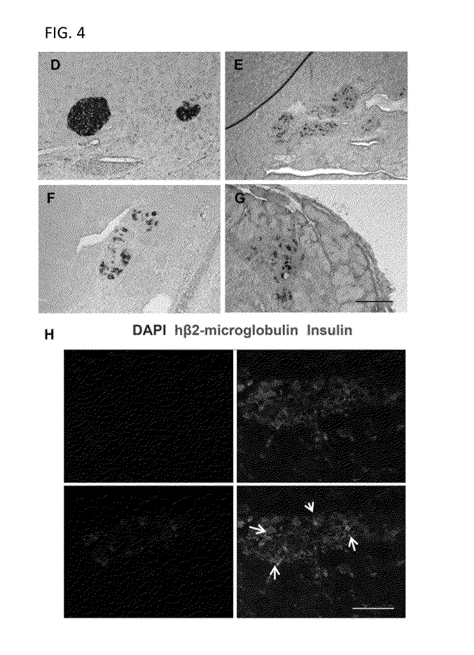

The inventors tried to determine if hBMSCs genetically modified to express PDX1 (hBMSCs-PDX1) were able to rescue diabetic mice. The cells were injected into the circulation of the diabetic mice (n=8) 7 day after STZ treatment. Four mice showed reduction of the hyperglycemia in the following week after transplantation (‘temporary reversed’), while 4 mice maintained severe hyperglycemia (‘unrescued’, FIG. 4A). Interestingly, the rescued mice maintained near-normoglycemic remission for 2-3 weeks, and then developed severe hyperglycemia again. All mice (‘temporary reversed’ and ‘unrescued’) survived at 6 weeks from transplantation, compared with the significant drop in survival rate of the diabetic mice (FIG. 4B, p<0.05), and gained significant weight compared with the diabetic control mice (FIG. 4C), indicating a better clinical outcome. Isolated pancreas from ‘temporary reversed’ as well as ‘unrescued’ mice analyzed for immunostaining against insulin showed reduction of insulin expression in the pancreatic islets compared (FIG. 4F,G) with healthy control mice (FIG. 4D) and similar to the STZ-treated diabetic mice (FIG. 4E). Staining for human beta-2 microglobulin clearly showed the engraftment of human cells in pancreases (FIG. 4H). In both groups (‘temporary reversed’ and ‘unrescued’), the inventors found none of vessel-like structures were differentiated from the hBMSCs-PDX1. A few transplanted cells expressing insulin in islet structures were noted as shown in FIG. 4H, implicating functional differentiation into β-cells.

### Example 20B

### Human Mesenchymal Stem Cell from Bone Marrow Alone Did not Induced Recovery of STZ-Induced Diabetes

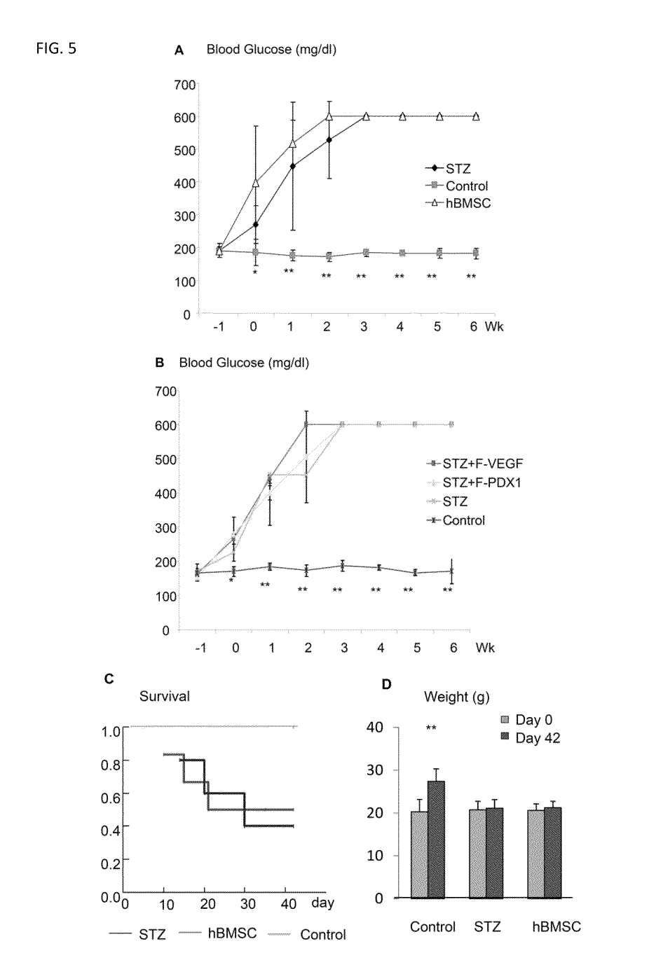

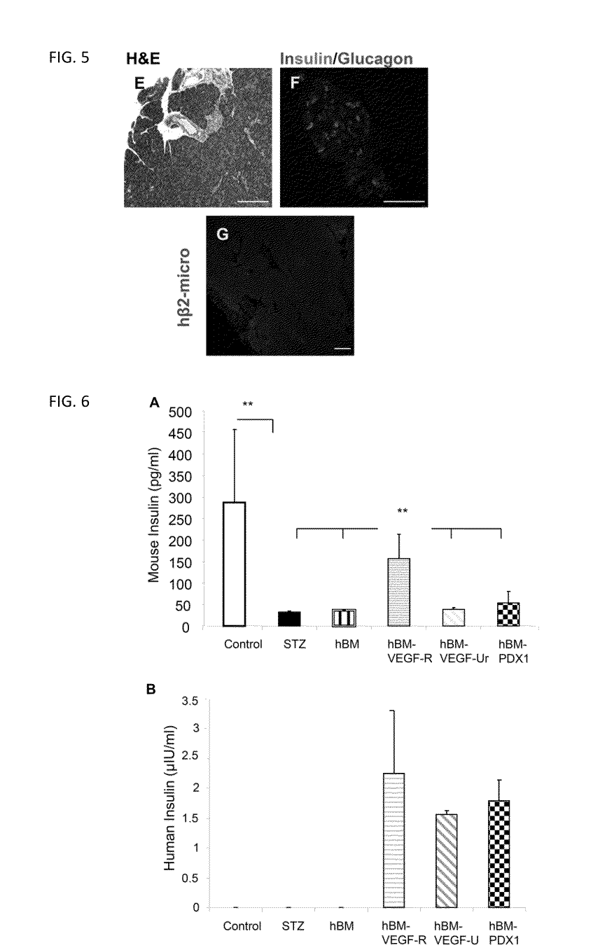

In contrast with the two above results, hBMSCs that were not genetically modified were not able to ameliorate the diabetes phenotype. Diabetic mice (n=6) treated with one intra-left ventricular injection of 1×10⁶ hBMSCs at day 7 from STZ injection continued to maintain severe hyperglycemia (FIG. 5A). Control experiments were carried on with fibroblasts expressing VEGF or PDX1 and did not show improvement of hyperglycemia after fibroblast transplantation (FIG. 5B). More than 50% of the mice treated with hBMSCs died before 6 weeks showing a survival rate similar to the STZ-induced diabetic mice and significantly lower than the healthy control mice (FIG. 5C, p<0.05). In addition survived mice that survived failed to gain weight (FIG. 5D) at 6 weeks post-transplantation. Histological examination showed severe alteration of the pancreatic islet morphology in this group (FIG. 5E) similar to the STZ-induced diabetic mice. The pancreatic islets of mice receiving hBMSC showed reduction in insulin expression with the characteristic inversion in the ratio insulin/glucagon cells (FIG. 5F). Further investigation showed poor engraftment of hBMSCs in the pancreas at 6 weeks after transplantation (FIG. 5G). Detection of human DNA in the mouse pancreas at 6 weeks post-transplantation was low (Table 3). The inventors were able to detect human DNA in two out of four tested mice with a lower concentration compared with the mice treated with hBMSC-VEGF, confirming a lower engraftment and/or survival of donor cell in the recipient pancreas.

TABLE 3
Human cell engraftment assayed by real-time PCR.

| Animal/Cells | Pancreas | Kidney | Liver |
|---|---|---|---|
| 1/hBMSC-VEGF | 0.2 ± 0.05 | ND | NA |
| 2/hBMSC-VEGF | 0.18 ± 0.07 | ND | NA |
| 3/hBMSC-VEGF | 0.025 ± 0.005 | 0.004 ± 0.001 | ND |
| 4/hBMSC-VEGF | 0.03 ± 0.007 | 0.015 ± 0.007 | ND |
| 1/hBMSC | 0.008 ± 0.0005 | ND | NA |
| 2/hBMSC | 0.0048 ± 0.001 | ND | NA |
| 3/hBMSC | ND | ND | ND |
| 4/hBMSC | ND | ND | NA |
| 1-3/no cells | ND | ND | NA |

Percentage of human DNA infused as cells.
NA, not assayed;
ND, not detected.
Mean ± SD

### Example 20C

### Contribution of Genetically Modified Human Bone Marrow Mesenchymal Stem Cells to β Cell Recovery: Endogenous Versus Transplant-Derived β-Cell Differentiation

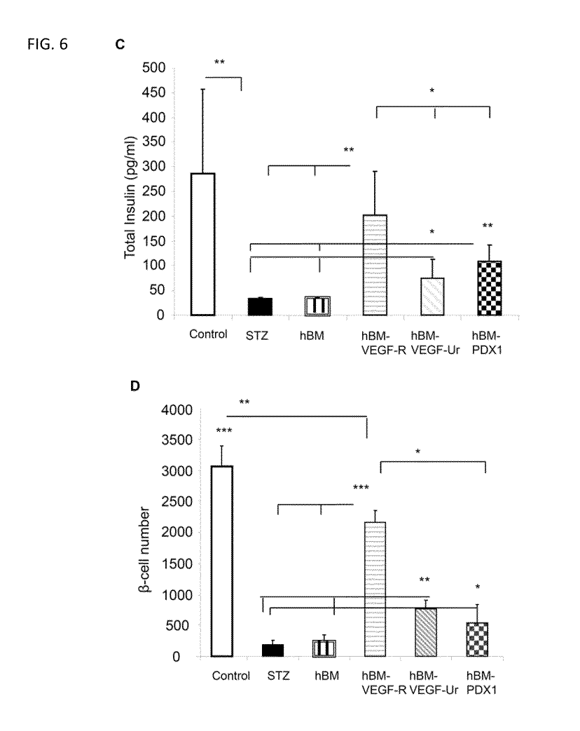

To evaluate the possible contribution of hBMSC-VEGF to the recovery of β-cells and whether the reversion of diabetes was secondary to a direct differentiation of hBMSC to β-cells or secondary to endogenous β-cell regeneration, the inventors measured both mouse and human serum insulin. Only the mice rescued by hBMSC-VEGF had significantly higher level of mouse insulin compared with that of diabetic mice and diabetic mice treated with hBMSC or hBMSC-PDX1 (FIG. 6A). However, more than half of the mice treated with hBMSC-VEGF and hBMSC-PDX1 (5 out of 9 and 5 out of 8, respectively), showed low but detectable level of human insulin, confirming de novo human BMSC differentiation into functional β-cells (FIG. 6B). The levels of total serum insulin were significantly higher in the mice treated with hBMSC-VEGF and hBMSC-PDX1 compared with the diabetic mice and the diabetic mice treated with hBMSC (FIG. 6C). The result showed clear correlation between the level of insulin and the number of β-cells, which was again higher in the mice treated with hBMSC-VEGF and hBMSC-PDX1 than two other groups (FIG. 6D). In addition, even unrescued groups of mice from hBMSC-VEGF and hBMSC-PDX1 with persistent hyperglycemia demonstrated significantly higher levels of total serum insulin and numbers of endogenous β-cells compared with other groups resulting in the overall better clinical outcomes. However, the group of mice with sustained near-normoglycemia remission, treated with hBMSC-VEGF, had the highest level of mouse insulin and β-cell numbers among the groups, suggesting that sustained reversion of diabetes was secondary to endogenous β-cell regeneration or recovery rather than transplant-derived β-cell differentiation.

### Example 21

### Endogenous β-Cell Recovery in Mouse Rescued by hBMSC-VEGF was Secondary to Activation of Insulin/IGF Signaling Pathway

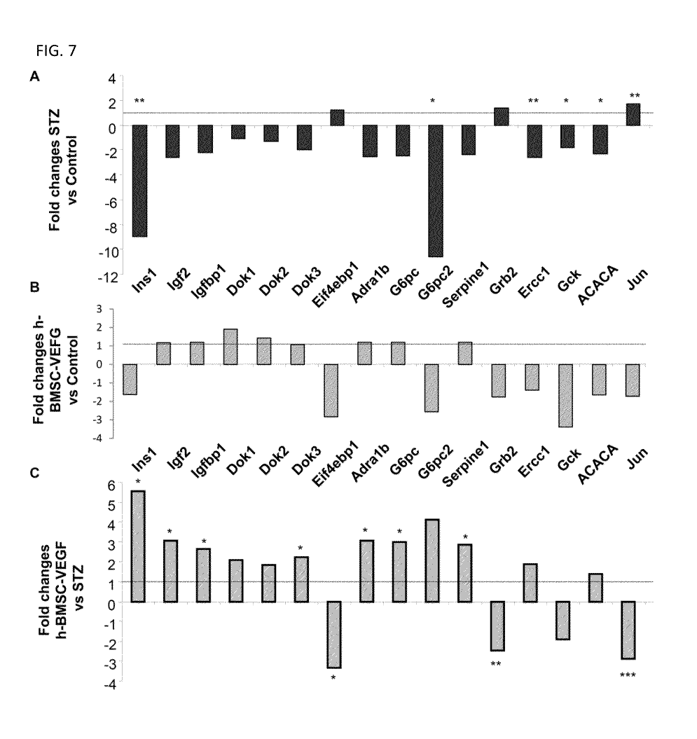

To evaluate the possible mechanism of endogenous β-cell recovery mediated by hBMSC-VEGF in the rescued mice the inventors compared the PCR array data of the pancreases from the healthy and diabetic age matched control mice. The inventors noted a clear trend of decreasing expression of mouse genes related with insulin receptor signaling pathway in pancreases of the diabetic mice compared with the healthy control (FIG. 7A). In the STZ-induced diabetic mice group, there was a significant decrease in expression of Insulin1, as expected. In addition, a significant decrease in gene expression of Excision repair cross-complementing rodent repair deficiency 1 (Ercc1), Glucokinase (Gck), and Acetyl-Coenzyme A carboxylase alpha (ACACA) with an increased expression of Jun. However, mice rescued by hBMSC-VEGF showed a similar PCR-array profile as seen in the healthy control mice without significant changes in gene expression (FIG. 7B). Interestingly, PCR array of the diabetic mice rescued by hBMSC-VEGF showed the significant up-regulation of the mouse genes involved in the insulin/IGF signaling pathway as compared to the diabetic mice (FIG. 7C). In particular, insulin was significantly upregulated in the rescued mice while it was downregulated in the STZ-induced diabetes mice as compared to the healthy control. Insulin receptor associate protein such as insulin growth factor 2 (IGF2), insulin growth factor binding protein 1 (Igfbp1), and Dok3 were significantly upregulated in the rescued mice. All target genes for phosphatidylinositol 3-kinase (PI-3K) pathway such as Adrenergic receptor alpha1d (Adra1d), glucose-6-phosphatase catalytic (G6 pc), glucose-6-phosphatase, catalytic, 2(G6pc2), and Serpine 1 were also upregulated while Eukaryotic translational initiation factor 2B, subunit 1 (Eif4ebp1), Growth factor receptor bound protein 2 (Grb2) and Jun were significantly downregulated as compared to the diabetic mice (FIG. 7C).

To further explore the molecular mechanisms for the reversion of diabetes and β-cell recovery/regeneration in diabetic mice treated with hBMSC-VEGF, pancreatic islets from control healthy mice, STZ-induced diabetic mice, and diabetic mice rescued by hBMSC-VEGF were examined using high resolution confocal imaging to assess insulin/IGF receptor/PI3-K downstream proteins. AKT protein was highly expressed in the pancreatic islets of healthy mice mostly on the plasma membranes (FIG. 8A, upper panel) while dramatically reduced in diabetic condition (FIG. 8A, intermediate panel). Interestingly, after injection of hBMSC-VEGF, pancreatic islets of rescued mice showed upregulation of AKT expression with distribution not only in the plasma membrane but also strongly in the cytoplasm (FIG. 8A, lower panel). Activation of insulin/IGF receptor/PI-3K/AKT pathway is associated to increase β-cell mass though activation of downstream proteins required for β-cell proliferation, differentiation and survival, such as PDX1 [27,28] and P27Kip1 [29,30]. Associated with preservation and/or regeneration of β-cell number and insulin secretion, mice rescued by hBMSC-VEGF showed a strong nuclear localization of PDX1 (FIG. 8B lower panel) similar to the control mice (FIG. 8B upper panel). In contrast PDX1 was detected in less β-cell nuclei of the diabetic mice (FIG. 8B, intermediate panel), with a staining that appears to be weaker. In addition, virtually all β-cell nuclei of the control mice were positive for p27^{Kip1} (FIG. 8C, upper panel), a cell cycle inhibitor protein negatively regulated through the PI-3K/AKT pathway. This is consistent with the slow β-cell replication in postnatal life [31]. After induction of diabetes most β-cell nuclei were positive for p27^{Kip1} (FIG. 8C, intermediate panel), suggesting a persistent inhibition of β-cell replication. In contrast the inventors detected a markedly decrease in immunostaining for p27^{Kip1} protein in the pancreatic islets of mice rescued by hBMSC-VEGF, with only isolated positive nuclei (FIG. 8C, lower panel), consistent with activation of β-cell replication.

The inventors next measured caspase 3 cleaved (c-CASP3) to β-cell assess apoptosis in relation to activation of the Insulin receptor/PI-3K/AKT pathway. Pancreatic islets from diabetic mice showed a dramatic increase in c-CASP3 compared with control and rescued mice (FIG. 8D), and the percent of c-CASP3-positive β-cells in diabetic mice rescued by hBMSC-VEGF was significantly lower compared to the diabetic mice and similar to the healthy control mice (FIG. 8E). This is consistent with anti-apoptotic signal mediated by activation of PI-3K pathway (FIG. 8F).

### REFERENCES

Alpert S, Hanahan D & Teitelman G 1988 Hybrid insulin genes reveal a developmental lineage for pancreatic endocrine cells and imply a relationship with neurons. Cell 53 295-308.
Atwater I, Goncalves A, Herchuelz A, Lebrun P, Malaisse W J, Rojas E & Scott A 1984 Cooling dissociates glucose-induced insulin release from electrical activity and cation fluxes in rodent pancreatic islets. Journal of Physiology 348 615-627.
Bonner-Weir S 2000 Life and death of the pancreatic beta cells. Trends in Endocrinology and Metabolism 11 375-378.
Bonner-Weir S 2001 β-cell turnover: its assessment and implications. Diabetes 50 (Suppl 1) S20-S24.
Bouwens L & Kloppel G 1996 Islet cell neogenesis in the pancreas. Virchows Archiv 427 553-560.
Cai J, Yu C, Liu Y, Chen S, Guo Y, Yong J, Lu W, Ding M & Deng H 2009 Generation of homogeneous PDX1C pancreatic progenitors from human ES cell-derived endoderm cells. Journal of Molecular Cell Biology 2 50-60.
Calderari S, Gangnerau M N, Thibault M, Meile M J, Kassis N, Alvarez C, Portha B & Serradas P 2007 Defective IGF2 and IGF1R protein production in embryonic pancreas precedes beta cell mass anomaly in the Goto-Kakizaki rat model of type 2 diabetes. Diabetologia 50 1463-1471.
Chang S, McKinsey T A, Zhang C L, Richardson J A, Hill J A & Olson E N 2004 Histone deacetylases 5 and 9 govern responsiveness of the heart to a subset of stress signals and play redundant roles in heart development. Molecular and Cellular Biology 24 8467-8476.
Choi K S, Shin J S, Lee J J, Kim Y S, Kim S B & Kim C W 2005 In vitro transdifferentiation of rat mesenchymal cells into insulin-producing cells by rat pancreatic extract. Biochemical and Biophysical Research Communications 330 1299-1305.
D'Amour K A, Bang A G, Eliazer S, Kelly O G, Agulnick A D, Smart N G, Moorman M A, Kroon E, Carpenter M K & Baetge E E 2006 Production of pancreatic hormone-expressing endocrine cells from human embryonic stem cells. Nature Biotechnology 24 1392-1401.
Dor Y, Brown J, Martinez O I & Melton D A 2004 Adult pancreatic beta-cells are formed by self-duplication rather than stem-cell differentiation. Nature 429 41-46.
Gao X, Song L, Shen K, Wang H, Niu W & Qin X 2008 Transplantation of bone marrow derived cells promotes pancreatic islet repair in diabetic mice. Biochemical and Biophysical Research Communications 371 132-137.

Hasegawa Y, Ogihara T, Yamada T, Ishigaki Y, Imai J, Uno K, Gao J, Kaneko K, Ishihara H, Sasano H et al. 2007 Bone marrow (BM) transplantation promotes beta-cell regeneration after acute injury through BM cell mobilization. Endocrinology 148 2006-2015.
Haumaitre C, Lenoir O & Scharfmann R 2008 Histone deacetylase inhibitors modify pancreatic cell fate determination and amplify endocrine progenitors. Molecular and Cellular Biology 28 6373-6383.
Hunziker E & Stein M 2000 Nestin-expressing cells in the pancreatic islets of Langerhans. Biochemical and Biophysical Research Communications 271 116-119.
Ianus A, Holz G G, Theise N D & Hussain M A 2003 In vivo derivation of glucose-competent pancreatic endocrine cells from bone marrow without evidence of cell fusion. Journal of Clinical Investigation 111 843-850.
Jensen J 2004 Gene regulatory factors in pancreatic development. Developmental Dynamics 229 176-200.
Johannesson M, Stahlberg A, Ameri J, Sand F W, Norrman K & Semb H 2009 FGF4 and retinoic acid direct differentiation of hESCs into PDX1-expressing foregut endoderm in a time- and concentration-dependent manner. PLoS ONE 4 e4794.
Jonas J C, Sharma A, Hasenkamp W, Ilkova H, Patané G, Laybutt R, Bonner-Weir S & Weir G C 1999 Chronic hyperglycemia triggers loss of pancreatic beta cell differentiation in an animal model of diabetes. Journal of Biological Chemistry 274 14112-14121.
Kabos P, Ehtesham M, Kabosova A, Black K L & Yu J S 2002 Generation of neural progenitor cells from whole adult bone marrow. Experimental Neurology 178 288-293.
Kim S Y, Lee S H, Kim B M, Kim E H, Min B H, Bendayan M & Park I S 2004 Activation of nestin-positive duct stem (NPDS) cells in pancreas upon neogenic motivation and possible cytodifferentiation into insulin-secreting cells from NPDS cells. Developmental Dynamics 230 1-11.
Kim S Y, Lee S, Hong S W, Min B H, Lee K U, Bendayan M & Park I S 2010 Nestin action during insulin-secreting cell differentiation. Journal of Histochemistry and Cytochemistry 58 567-576.
Kuo C Y, Herrod H G & Burghen G A 1992 Formation of pseudoislets from human pancreatic cultures. Pancreas 7 320-325.
Lakey J R, Mirbolooki M & Shapiro A M 2006 Current status of clinical islet cell transplantation. Methods in Molecular Biology 333 47-104.
Lefebvre B, Belaich S, Longue J, Vandewalle B, Oberholzer J, Gmyr V, Pattou F & Kerr-Conte J 2009 50-AZA induces Ngn3 expression and endocrine differentiation in the PANC-1 human ductal cell line. Biochemical and Biophysical Research Communications 391 305-309.
Lendahl U, Zimmerman L B & McKay R D 1990 CNS stem cells express a new class of intermediate filament protein. Cell 60 585-595.
Madsen O D, Jensen J, Petersen H V, Pedersen E E, Oster A, Andersen F G, Jorgensen M C, Jensen P B, Larsson L I & Serup P 1997 Transcription factors contributing to the pancreatic beta-cell phenotype. Hormone and Metabolic Research 29 265-270.
Matsuoka M, Osada T, Yoshida-Matsuoka J, Ikai A, Ichikawa M, Norita M & Costanzo R M 2002 A comparative immunocytochemical study of development and regeneration of chemosensory neurons in the rat vomeronasal system. Brain Research 946 52-63.
Micallef S J, Janes M E, Knezevic K, Davis R P, Elefanty A G & Stanley E G 2005 Retinoic acid induces Pdx1-positive endoderm in differentiating mouse embryonic stem cells. Diabetes 54 301-305.
Morshead C M, Reynolds B A, Craig C G, McBurney M W, Staines W A, Morassutti D, Weiss S & van der Kooy D 1994 Neural stem cells in the adult mammalian forebrain: a relatively quiescent subpopulation of subependymal cells. Neuron 13 1071-1082.
Novitch B G, Wichterle H, Jessell T M & Sockanathan S 2003 A requirement for retinoic acid-mediated transcriptional activation in ventral neural patterning and motor neuron specification. Neuron 40 81-95.
Ostrom M, Loffler K A, Edfalk S, Selander L, Dahl U, Ricordi C, Jeon J, Correa-Medina M, Diez J & Edlund H 2008 Retinoic acid promotes the generation of pancreatic endocrine progenitor cells and their further differentiation into beta-cells. PLoS ONE 3 e2841.
Reynolds B A & Weiss S 1996 Clonal and population analyses demonstrate that an EGF-responsive mammalian embryonic CNS precursor is a stem cell. Developmental Biology 175 1-13.
Sander M & German M S 1997 The beta cell transcription factors and development of the pancreas. Journal of Molecular Medicine 75 327-340.
Shapiro A M, Ricordi C, Hering B J, Auchincloss H, Lindblad R, Robertson R P, Secchi A, Brendel M D, Berney T, Brennan D C et al. 2006 International trial of the Edmonton protocol for islet transplantation. New England Journal of Medicine 355 1318-1330.
Shi Y, Hou L, Tang F, Jiang W, Wang P, Ding M & Deng H 2005 Inducing embryonic stem cells to differentiate into pancreatic beta cells by a novel three-step approach with activin A and all-trans retinoic acid. Stem Cells 23 656-662.
Shiroi A, Yoshikawa M, Yokota H, Fukui H, Ishizaka S, Tatsumi K & Takahashi Y 2002 Identification of insulin-producing cells derived from embryonic stem cells by zinc-chelating dithizone. Stem Cells 20 284-292.
Talmor M, Mirza A, Turley S, Mellman I, Hoffman L A & Steinman R M 1998 Generation or large numbers of immature and mature dendritic cells from rat bone marrow cultures. European Journal of Immunology 28 811-817.
Tomita S, Li R K, Weisel R D, Mickle D A, Kim E J, Sakai T & Jia Z Q 1999 Autologous transplantation of bone marrow cells improves damaged heart function. Circulation 100 II247-II256.
Vaes B L, Lute C, van derWoning S P, Piek E, Vermeer J, Blom H J, Mathers J C, Muller M, de Groot L C & Steegenga W T 2009 Inhibition of methylation decreases osteoblast differentiation via a non-DNA-dependent methylation mechanism. Bone 46 514-523.
Xu J, Lu Y, Ding F, Zhan X, Zhu M & Wang Z 2007 Reversal of diabetes in mice by intrahepatic injection of bone-derived GFP-murine mesenchymal stem cells infected with the recombinant retrovirus-carrying human insulin gene. World Journal of Surgery 31 1872-1882.
Yashpal N K, Li J & Wang R 2004 Characterization of c-Kit and nestin expression during islet cell development in the prenatal and postnatal rat pancreas. Developmental Dynamics 229 813-825.
Ye D Z, Tai M H, Linning K D, Szabo C & Olson L K 2006 MafA expression and insulin promoter activity are induced by nicotinamide and related compounds in INS-1 pancreatic beta-cells. Diabetes 55 742-750.
Zeng Z, Yuan X, Liu G, Zeng X, Ng H, Chen H, Jiang T, Akasaki Y, Kessey K, Black K L et al. 2007 Manipulation of proliferation and differentiation of human bone marrow-derived neural stem cells in vitro and in vivo. Journal of Neuroscience Research 85 310-320.
Zhang C L, McKinsey T A, Chang S, Antos C L, Hill J A & Olson E N 2002 Class II histone deacetylases act as signal-responsive repressors of cardiac hypertrophy. Cell 110 479-488.
Zhao M, Amiel S A, Ajami S, Jiang J, Rela M, Heaton N & Huang G C 2008 Amelioration of streptozotocin-induced diabetes in mice with cells derived from human marrow stromal cells. PLoS ONE 3 e2666.
Zulewski H, Abraham E J, Gerlach M J, Daniel P B, Moritz W, Muller B, Vallejo M, Thomas M K & Habener J F 2001 Multipotential nestin-positive stem cells isolated from adult pancreatic islets differentiate ex vivo into pancreatic endocrine, exocrine, and hepatic phenotypes.
1. Ianus A, Holz G G, Theise N D, Hussain M A (2003) In vivo derivation of glucose-competent pancreatic endocrine cells from bone marrow without evidence of cell fusion. J Clin Invest 111: 843-850.
2. Zhao M, Amiel S A, Ajami S, Jiang J, Rela M, et al. (2008) Amelioration of streptozotocin-induced diabetes in mice with cells derived from human marrow stromal cells. PLoS One 3: e2666.
3. Hess D, Li L, Martin M, Sakano S, Hill D, et al. (2003) Bone marrow-derived stem cells initiate pancreatic regeneration. Nat Biotechnol 21: 763-770.
4. Alvarez S S, Jimenez L M, Murillo A Z, Gomez I G, Ligero J M, et al. (2006) A new approach for bone marrow-derived stem cells intrapancreatic autotransplantation in diabetic rats. Microsurgery 26: 539-542.
5. Banerjee M, Kumar A, Bhonde R R (2005) Reversal of experimental diabetes by multiple bone marrow transplantation. Biochem Biophys Res Commun 328: 318-325.
6. ksadHasegawa Y, Ogihara T, Yamada T, Ishigaki Y, Imai J, et al. (2007) Bone marrow (BM) transplantation promotes beta-cell regeneration after acute injury through BM cell mobilization. Endocrinology 148: 2006-2015.
7. Lee R H, Seo M J, Reger R L, Spees J L, Pulin A A, et al. (2006) Multipotent stromal cells from human marrow home to and promote repair of pancreatic islets and renal glomeruli in diabetic NOD/scid mice. Proc Natl Acad Sci USA 103: 17438-17443.
8. Urban V S, Kiss J, Kovacs J, Gocza E, Vas V, et al. (2008) Mesenchymal stem cells cooperate with bone marrow cells in therapy of diabetes. Stem Cells 26: 244-253.
9. Fiorina P, Jurewicz M, Augello A, Vergani A, Dada S, et al. (2009) Immunomodulatory function of bone marrow-derived mesenchymal stem cells in experimental autoimmune type 1 diabetes. J Immunol 183: 993-1004.
10. Ezquer F E, Ezquer M E, Parrau D B, Carpio D, Yanez A J, et al. (2008) Systemic administration of multipotent mesenchymal stromal cells reverts hyperglycemia and prevents nephropathy in type 1 diabetic mice. Biol Blood Marrow Transplant 14: 631-640.
11. Ezquer F, Ezquer M, Simon V, Conget P (2011) The antidiabetic effect of MSCs is not impaired by insulin prophylaxis and is not improved by a second dose of cells. PLoS One 6: e16566.
12. Milanesi A, Lee J W, Xu Q, Perin L, Yu J S (2011) Differentiation of nestin-positive cells derived from bone marrow into pancreatic endocrine and ductal cells in vitro. J Endocrinol 209: 193-201.
13. Kabos P, Ehtesham M, Kabosova A, Black K L, Yu J S (2002) Generation of neural progenitor cells from whole adult bone marrow. Exp Neurol 178: 288-293.
14. Zeng Z, Yuan X, Liu G, Zeng X, Ng H, et al. (2007) Manipulation of proliferation and differentiation of human bone marrow-derived neural stem cells in vitro and in vivo. J Neurosci Res 85: 310-320.
15. Tang D Q, Cao L Z, Burkhardt B R, Xia C Q, Litherland S A, et al. (2004) In vivo and in vitro characterization of insulin-producing cells obtained from murine bone marrow. Diabetes 53: 1721-1732.
16. Chen L B, Jiang X B, Yang L (2004) Differentiation of rat marrow mesenchymal stem cells into pancreatic islet beta-cells. World J Gastroenterol 10: 3016-3020.
17. Karnieli O, Izhar-Prato Y, Bulvik S, Efrat S (2007) Generation of insulin-producing cells from human bone marrow mesenchymal stem cells by genetic manipulation. Stem Cells 25: 2837-2844.
18. Fujitani Y, Fujitani S, Boyer D F, Gannon M, Kawaguchi Y, et al. (2006) Targeted deletion of a cis-regulatory region reveals differential gene dosage requirements for Pdx1 in foregut organ differentiation and pancreas formation. Genes Dev 20: 253-266.
19. Holland A M, Gonez L J, Naselli G, Macdonald R J, Harrison L C (2005) Conditional expression demonstrates the role of the homeodomain transcription factor Pdx1 in maintenance and regeneration of beta-cells in the adult pancreas. Diabetes 54: 2586-2595.
20. Brissova M, Shostak A, Shiota M, Wiebe P O, Poffenberger G, et al. (2006) Pancreatic islet production of vascular endothelial growth factor—a is essential for islet vascularization, revascularization, and function. Diabetes 55: 2974-2985.
21. Nikolova G, Jabs N, Konstantinova I, Domogatskaya A, Tryggvason K, et al. (2006) The vascular basement membrane: a niche for insulin gene expression and Beta cell proliferation. Dev Cell 10: 397-405.
22. Nicholson J M, Arany E J, Hill D J (2010) Changes in islet microvasculature following streptozotocin-induced beta-cell loss and subsequent replacement in the neonatal rat. Exp Biol Med (Maywood) 235: 189-198.
23. Christofori G, Naik P, Hanahan D (1995) Vascular endothelial growth factor and its receptors, flt-1 and flk-1, are expressed in normal pancreatic islets and throughout islet cell tumorigenesis. Mol Endocrinol 9: 1760-1770.
24. Chae H Y, Lee B W, Oh S H, Ahn Y R, Chung J H, et al. (2005) Effective glycemic control achieved by transplanting non-viral cationic liposome-mediated VEGF-transfected islets in streptozotocin-induced diabetic mice. Exp Mol Med 37: 513-523.
25. Narang A S, Sabek O, Gaber A O, Mahato R I (2006) Co-expression of vascular endothelial growth factor and interleukin-1 receptor antagonist improves human islet survival and function. Pharm Res 23: 1970-1982.
26. Malek A, Catapano C V, Czubayko F, Aigner A (2010) A sensitive polymerase chain reaction-based method for detection and quantification of metastasis in human xenograft mouse models. Clin Exp Metastasis 27: 261-271.
27. Kulkarni R N, Jhala U S, Winnay J N, Krajewski S, Montminy M, et al. (2004) PDX-1 haploinsufficiency limits the compensatory islet hyperplasia that occurs in response to insulin resistance. J Clin Invest 114: 828-836.
28. Kitamura T, Nakae J, Kitamura Y, Kido Y, Biggs W H, 3rd, et al. (2002) The forkhead transcription factor Foxol links insulin signaling to Pdx1 regulation of pancreatic beta cell growth. J Clin Invest 110: 1839-1847.
29. Jiang B H, Zheng J Z, Aoki M, Vogt P K (2000) Phosphatidylinositol 3-kinase signaling mediates angiogenesis and expression of vascular endothelial growth factor in endothelial cells. Proc Natl Acad Sci USA 97: 1749-1753.
30. Tanabe K, Liu Z, Patel S, Doble B W, Li L, et al. (2008) Genetic deficiency of glycogen synthase kinase-3beta corrects diabetes in mouse models of insulin resistance. PLoS Biol 6: e37.
31. Folli F, Okada T, Perego C, Gunton J, Liew C W, et al. (2011) Altered insulin receptor signalling and beta-cell cycle dynamics in type 2 diabetes mellitus. PLoS One 6: e28050.
32. Hasegawa Y, Ogihara T, Yamada T, Ishigaki Y, Imai J, et al. (2007) Bone marrow (BM) transplantation promotes beta-cell regeneration after acute injury through BM cell mobilization. Endocrinology 148: 2006-2015.
33. Esser S, Wolburg K, Wolburg H, Breier G, Kurzchalia T, et al. (1998) Vascular endothelial growth factor induces endothelial fenestrations in vitro. J Cell Biol 140: 947-959.
34. Issbrucker K, Marti H H, Hippenstiel S, Springmann G, Voswinckel R, et al. (2003) p38 MAP kinase—a molecular switch between VEGF-induced angiogenesis and vascular hyperpermeability. FASEB J 17: 262-264.
35. Lin H, Shabbir A, Molnar M, Yang J, Marion S, et al. (2008) Adenoviral expression of vascular endothelial growth factor splice variants differentially regulate bone marrow-derived mesenchymal stem cells. J Cell Physiol 216: 458-468.
36. Matsumoto R, Omura T, Yoshiyama M, Hayashi T, Inamoto S, et al. (2005) Vascular endothelial growth factor-expressing mesenchymal stem cell transplantation for the treatment of acute myocardial infarction. Arterioscler Thromb Vasc Biol 25: 1168-1173.
37. Al-Khaldi A, Eliopoulos N, Martineau D, Lejeune L, Lachapelle K, et al. (2003) Postnatal bone marrow stromal cells elicit a potent VEGF-dependent neoangiogenic response in vivo. Gene Ther 10: 621-629.
38. Iwashita N, Uchida T, Choi J B, Azuma K, Ogihara T, et al. (2007) Impaired insulin secretion in vivo but enhanced insulin secretion from isolated islets in pancreatic beta cell-specific vascular endothelial growth factor-A knock-out mice. Diabetologia 50: 380-389.
39. Nakayama S, Uchida T, Choi J B, Fujitani Y, Ogihara T, et al. (2009) Impact of whole body irradiation and vascular endothelial growth factor-A on increased beta cell mass after bone marrow transplantation in a mouse model of diabetes induced by streptozotocin. Diabetologia 52: 115-124.
40. Sakata N, Chan N K, Chrisler J, Obenaus A, Hathout E (2010) Bone marrow cell cotransplantation with islets improves their vascularization and function. Transplantation 89: 686-693.
41. Duffy G P, Ahsan T, O'Brien T, Barry F, Nerem R M (2009) Bone marrow-derived mesenchymal stem cells promote angiogenic processes in a time- and dose-dependent manner in vitro. Tissue Eng Part A 15: 2459-2470.
42. Tamama K, Sen C K, Wells A (2008) Differentiation of bone marrow mesenchymal stem cells into the smooth muscle lineage by blocking ERK/MAPK signaling pathway. Stem Cells Dev 17: 897-908.
43. Yue W M, Liu W, Bi Y W, He X P, Sun W Y, et al. (2008) Mesenchymal stem cells differentiate into an endothelial phenotype, reduce neointimal formation, and enhance endothelial function in a rat vein grafting model. Stem Cells Dev 17: 785-793.
44. Gong Z, Niklason L E (2008) Small-diameter human vessel wall engineered from bone marrow-derived mesenchymal stem cells (hMSCs). FASEB J 22: 1635-1648.
45. Forte A, Finicelli M, Mattia M, Berrino L, Rossi F, et al. (2008) Mesenchymal stem cells effectively reduce surgically induced stenosis in rat carotids. J Cell Physiol 217: 789-799.
46. Quevedo H C, Hatzistergos K E, Oskouei B N, Feigenbaum G S, Rodriguez J E, et al. (2009) Allogeneic mesenchymal stem cells restore cardiac function in chronic ischemic cardiomyopathy via trilineage differentiating capacity. Proc Natl Acad Sci USA 106: 14022-14027.
47. Wang Q, Jin T (2009) The role of insulin signaling in the development of beta-cell dysfunction and diabetes. Islets 1: 95-101.
48. Matsuoka T A, Kaneto H, Miyatsuka T, Yamamoto T, Yamamoto K, et al. (2010) Regulation of MafA expression in pancreatic beta-cells in db/db mice with diabetes. Diabetes 59: 1709-1720.
49. Assmann A, Hinault C, Kulkarni R N (2009) Growth factor control of pancreatic islet regeneration and function. Pediatr Diabetes 10: 14-32.
50. Tuttle R L, Gill N S, Pugh W, Lee J P, Koeberlein B, et al. (2001) Regulation of pancreatic beta-cell growth and survival by the serine/threonine protein kinase Aktl/PKBalpha. Nat Med 7: 1133-1137.
51. Gu Y, Lindner J, Kumar A, Yuan W, Magnuson M A (2011) Rictor/mTORC2 is essential for maintaining a balance between beta-cell proliferation and cell size. Diabetes 60: 827-837.
52. Andjelkovic M, Alessi D R, Meier R, Fernandez A, Lamb N J, et al. (1997) Role of translocation in the activation and function of protein kinase B. J Biol Chem 272: 31515-31524.
53. Brazil D P, Hemmings B A (2001) Ten years of protein kinase B signalling: a hard Akt to follow. Trends Biochem Sci 26: 657-664.
54. Federici M, Hribal M, Perego L, Ranalli M, Caradonna Z, et al. (2001) High glucose causes apoptosis in cultured human pancreatic islets of Langerhans: a potential role for regulation of specific Bcl family genes toward an apoptotic cell death program. Diabetes 50: 1290-1301.
55. Gratton J P, Morales-Ruiz M, Kureishi Y, Fulton D, Walsh K, et al. (2001) Akt down-regulation of p38 signaling provides a novel mechanism of vascular endothelial growth factor-mediated cytoprotection in endothelial cells. J Biol Chem 276: 30359-30365.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A method, comprising:
providing a nestin-positive bone marrow stem cell (BMSC);
culturing the nestin-positive BMSC in a first culture medium for chromatin remodeling by contacting the cell with 5-azacytidine (5-AZA) and contacting the cell with trichostatin (TSA);
culturing the cell in a second culture medium for cell induction;
culturing the cell in a third culture medium to differentiate the cell;
culturing the cell in a forth culture medium to mature the cell into a pancreatic precursor cell or an insulin-producing cell.

2. The method of claim 1, wherein the first culture medium comprises one or more agents selected from the group consisting of KO-DMEM, β-mercaptoethanol, L-glutamine, non-essential amino acid, BSA, N2 supplement, B27 supplement, heparin, bFGF, EGF, and combinations thereof.

3. The method of claim 1, wherein the second medium comprises one or more agents from the first culture medium and an agent selected from the group consisting of DMEM, low glucose, ITS, RA and combinations thereof.

4. The method of claim 1, wherein the third medium comprises one or more agents from the first culture medium and an agent selected from the group consisting of DMEM, high glucose and combinations thereof.

5. The method of claim 1, wherein the fourth medium comprises one or more agents from the first culture medium and an agent selected from the group consisting of DMEM, high glucose, nicotinamide, and combinations thereof.

6. The method of claim 1, wherein the fourth culture medium does not contain bFGF or EGF.

7. The method of claim 1, wherein the insulin-producing cell is an insulin producing β-cell or a β-islet cell.

8. A method, comprising:
providing a bone marrow stem cell (BMSC); and
transfecting the BMSC with one or more virus vectors encoding PDX1, VEGF, or both to produce a VEGF and/or PDX1 expressing BMSC.

9. The method of claim 8, wherein the virus is an adenovirus.

10. The method of claim 8, wherein the BMSC is obtained by:
providing bone marrow aspirate;
culturing a nucleated cell from the bone marrow aspirate;
separating a non-adherent cell from an adherent cell;
culturing the adherent cell;
harvesting a bone marrow stem cell;
and expanding the bone marrow stem cell.

11. The method of claim 10, wherein the nucleated cell from the bone marrow aspirate is cultured in a medium comprising a component selected from the group consisting of Afla-Men, fetal bovine serum, glutamine, penicillin, streptomycin and combinations thereof.

12. A method, comprising:
providing a bone marrow stem cell (BMSC) expressing VEGF, a BMSC expressing PDX1 or a BMSC expressing both VEGF and PDX1; and
administering the BMSC to a subject in need of treatment for diabetes or ameliorating a symptom of diabetes to treat diabetes or ameliorate the symptom of diabetes.

13. The method of claim 12, wherein:
the BMSC expressing VEGF, the BMSC expressing PDX1 or the BMSC expressing both VEGF and PDX1 is produced by:
isolating a BMSC from a bone marrow sample from the subject; and
artificially increasing the expression of VEGF and/or PDX1 in the BMSC.

14. The method of claim 12, wherein administering is via intravenous injection of BMSC expressing of VEGF and/or PDX1 into the circulation of the subject.

15. A method, comprising:
providing a pancreatic precursor cell or an insulin-producing cell differentiated from a bone marrow stem cell (BMSC); and
administering the pancreatic precursor cell or the insulin-producing cell differentiated from a BMSC to a subject in need of treatment for diabetes or ameliorating a symptom of diabetes to treat diabetes or ameliorate the symptom of diabetes.

16. The method of claim 15, further comprising:
obtaining bone marrow from the subject; and
producing the pancreatic precursor cell or the insulin-producing cell from the bone marrow.

17. The method of claim 15, wherein administering is via intra-hepatic or subcutaneous transplantation.

18. The method of claim 15, wherein the insulin-producing cell is an insulin-producing β-cell or a β-islet cell.

19. The method of claim 16, wherein producing the pancreatic precursor cell or an insulin-producing cell from the bone marrow is by:
producing a nestin-positive bone marrow stem cell (BMSC) from the bone marrow;
culturing the nestin-positive BMSC in a first culture medium for chromatin remodeling by contacting the cell with 5-azacytidine (5-AZA) and contacting the cell with trichostatin (TSA);
culturing the cell in a second culture medium for cell induction;
culturing the cell in a third culture medium to differentiate the cell;
culturing the cell in a forth culture medium to mature the cells into a pancreatic precursor cell or an insulin-producing beta-cell.

20. The method of claim 19, wherein the first culture medium comprises one or more agents selected from the group consisting of KO-DMEM, β-mercaptoethanol, L-glutamine, non-essential amino acid, BSA, N2 supplement, B27 supplement, heparin, bFGF, EGF, and combinations thereof.

21. The method of claim 19, wherein the second medium comprises one or more agents from the first culture medium and an agent selected from the group consisting of DMEM, low glucose, ITS, RA and combinations thereof.

22. The method of claim 19, wherein the third medium comprises one or more agents from the first culture medium and an agent selected from the group consisting of DMEM, high glucose and combinations thereof.

23. The method of claim 19, wherein the fourth medium comprises one or more agents from the first culture medium and an agent selected from the group consisting of DMEM, high glucose, nicotinamide, and combinations thereof.
